(19) United States
(12) Patent Application Publication
(10) Pub. No.: US 2026/0200820 A1
(43) Pub. Date: Jul. 16, 2026

(54) A REACTIVE SEPARATION PROCESS FOR CARBON DIOXIDE CAPTURE FROM FLUE GAS

(71) Applicant: UNIVERSITY OF SOUTHERN CALIFORNIA, Los Angeles, CA (US)

(72) Inventors: Theodore T. TSOTSIS, Los Angeles, CA (US); Kristian JESSEN, Los Angeles, CA (US); Fatemeh SADAT-ZEBARJAD, Los Angeles, CA (US); Jingwen GONG, Los Angeles, CA (US); Mohammad BAZMI, Los Angeles, CA (US); Linghao ZHAO, Los Angeles, CA (US)

(73) Assignee: UNIVERSITY OF SOUTHERN CALIFORNIA, Los Angeles, CA (US)

(21) Appl. No.: 19/131,401

(22) PCT Filed: Sep. 25, 2023
(86) PCT No.: PCT/US2023/033635

§ 371 Date: May 20, 2025

Related U.S. Application Data
(60) Provisional application No. 63/427,482, filed on Nov. 23, 2022.

(51) Int. Cl. C07C 29/152; B01D 69/04; B01D 71/02; B01J 23/06; B01J 23/80; C01B 32/40

(52) U.S. Cl. CPC C07C 29/152; B01D 69/04; B01D 71/02; B01J 23/06; B01J 23/80; C01B 32/40

(57) ABSTRACT

A system for converting CO₂ to methanol includes a reverse water gas shift (“RWGS”) reactor configured to receive a first CO₂ stream and a hydrogen gas stream under a sufficient temperature and a sufficient pressure for an RWGS reaction to proceed. The RWGS reactor outputs an exit stream that includes CO. The system also includes a heat exchanger/condenser in fluid communication with the RWGS reactor configured to remove water from products of the RWGS reaction to form a dried exit stream that includes CO; and a membrane contactor reactor configured to receive a combination of hydrogen, CO₂, and the dried exit stream. The membrane contactor reactor also configured to output a first output stream including methanol dissolved in a sweep liquid and a second output stream including gaseous H₂, gaseous CO, gaseous CO₂, and gaseous methanol.

### CROSS-REFERENCE TO RELATED APPLICATIONS

This application claims the benefit of U.S. provisional application Ser. No. 63/427,482 filed Nov. 23, 2022, the disclosure of which is hereby incorporated in its entirety by reference herein.

### STATEMENT REGARDING FEDERALLY SPONSORED RESEARCH OR DEVELOPMENT

This invention was made with government support under Grant No(s). DE-06-USC-21-10 and CBET-1705180, awarded by the Department of Energy (DOE) and National Science Foundation (NSF), respectively. The government has certain rights in the invention.”

### TECHNICAL FIELD

In at least one aspect, the present invention is related to methods and systems for separating carbon dioxide from flue gas.

### BACKGROUND

The continued reliance on fossil fuels like coal, oil and natural gas, has resulted in increased CO₂ emissions to the point where they have become today a global concern [3]. Long-term, the solution to this technical challenge is to replace the fossil fuels with alternative renewable energy sources (e.g., solar, geothermal, wind-power, etc.). In the interim period though, it is important that we separate, capture, and sequester the CO₂ generated so that it does not end-up as emissions to the atmosphere. Current CO₂ capture and storage (CCS) processes are both capital- and energy-intensive, however. So, a focus in recent years has been on developing technologies, known as carbon capture and utilization (CCU) processes, that utilize the CO₂ captured to convert it into fuels and chemicals, the motivation here being through the sale of such products to be able to defray, at least partially, some of the significant costs associated with CCS [4]. For such technologies to have an impact on addressing the CO₂ emissions challenge, the chemical(s) produced must have widespread use and significant market potential. One such chemical is MeOH whose potential production from waste CO₂ has been studied in recent years [5].

Direct catalytic conversion of CO₂ into MeOH has been receiving increased attention [6,7] with a number of catalysts being prepared and studied. The conventional Cu/ZnO/Al₂O₃ catalyst continues to be the most widely utilized catalyst formulation for converting CO₂ into MeOH because of its good activity and low cost. However, this catalyst was originally developed for the conversion of CO-rich syngas mixtures into MeOH and is, therefore, not optimized for the conversion of pure CO₂ or CO₂-rich mixtures. Current challenges include improving its activity at low temperatures, overcoming catalyst deactivation, and minimizing by-product formation [8,9].

A number of research groups have modified the conventional Cu—Zn-based catalyst by substituting in its formulation the Al₂O₃ support with other trivalent or tetravalent metal oxides. One of the most studied such oxides is ZrO₂ [8,10], which has a weaker hydrophilic character compared to Al₂O₃. This is reported to enhance the dispersion and stability of Cu, and to impede the adsorption of water [10]. It has also been reported that the substitution of Al₂O₃ by ZrO₂ increases the basicity of the catalyst [10] which, in turn, favors the selectivity of MeOH during MeS from CO₂-rich syngas mixtures.

Non-Cu type catalysts have also been studied employing noble metals, primarily Pd [11-13] and to a lesser extent, Au [14,15]. Pd is very active for the hydrogenation of CO₂, with its selectivity to MeOH depending on the type of support and promoters utilized [13]. Pd, when supported on ZnO, forms a bimetallic PdZn alloy which acts as the active phase for the selective production of MeOH [13]. The use of non-noble metals (Cu, Co, and Fe) supported on Mo₂C as active catalysts for the selective hydrogenation of CO₂ into MeOH under mild conditions (135-200° C. in a liquid solvent of 1,4-dioxane) was reported by Chen et al. [16]. The Mo₂C served as a support and co-catalyst for the reaction. Using pure Mo₂C, MeOH was the main product at 135° C., while MeOH, ethanol, and C₂₊ hydrocarbon compounds were produced at 200° C. The addition of Cu to the Mo₂C improved the production of MeOH, while the addition of Co and Fe only increased the production of C₂₊ hydrocarbons [16]. 1,4-dioxane served as a liquid solvent due to its high solubility toward MeOH, relatively high boiling point, and stability during the reaction 73 [17].

There have also been several efforts by industrial groups for the direct conversion of CO₂ into MeOH. One of the earlier industrial-scale processes to convert CO₂ into MeOH was developed by Lurgi AG in the 1990s [18,19]. It is a two-stage process: A gas mixture consisting of ~20% carbon oxides (CO+CO₂) in H₂ is pre-converted in an adiabatic packed-bed reactor (PBR) in a once-through operation before entering a MeS loop consisting of a steam producing, non-isothermal reactor. In both reactors, MeOH fonnation and the water gas shift (WGS) reaction proceed simultaneously, and MeOH and water are separated from the feed of the second reactor to increase the MeOH conversion.

An investigation of a single-stage pilot-plant to convert CO₂ into MeOH was conducted by Ponzen et al. [7]. They reported a single-pass conversion of 30-40% over the commercial Cu/ZnO catalyst with a feed H₂/CO₂ ratio equal to 3 at a temperature of 250° C. and a pressure of 70-80 bar. Specht and coworkers [20] studied the conversion at atmospheric pressure conditions of CO₂ and hydrogen into MeOH in a bench-scale PBR over a Cu/ZnO catalyst and reported a 23% conversion per pass. A lab-scale CO₂ into MeOH conversion set-up was developed by Morgan and Acker [21]: The hydrogen for the reaction is produced on-site from purified water via electrolysis. The produced MeOH and water are separated at the exit of the reactor, and the unreacted gas is recycled and mixed with the fresh CO₂ before the MeS loop. They report the overall first thermodynamic law conversion efficiency of MeOH production to be low (~16.5%) [21].

A two-stage process (named CAMERE) for C₀₂ hydrogenation into MeOH was developed by the Korean Institute of Science and Technology [22,23]. The first stage is a reverse water gas shift reactor (RWGSR) that operates at high temperatures, 600-700° C., with the goal of converting CO₂ into CO (conversion efficiency of ~60%). The exit stream from the RWGSR, after removal of the water produced, is then fed into the second stage MeS reactor. Although the MeOH conversion was increased due to the increase in CO content of the feed and water removal before the MeS reactor, the reported overall space time yields (kg lcat⁻¹ h⁻¹), not taking into account the catalyst mass in the RWGSR, are almost the same with the values reported for the direct CO₂ hydrogenation pilot plants [7,20,24].

In 2014, Carbon Recycling International (CRI) reported [25] the operation of a commercial-scale CO₂ to MeOH plant in Svartsengi, Iceland. The plant uses a conventional Cu/ZnO-catalyst and operates at 250° C. and 100 atm. It utilizes 5600 tons/per year of CO₂ released by a nearby geothernal power plant to produce 4000 tons/year of MeOH. The H₂ used in the process is produced by an alkaline electrolysis unit [26]. We do not know of any other commercial plant presently producing MeOH from pure CO₂ feeds.

In summary, there is a lot of interest today in finding ways to beneficially utilize waste CO₂, and its conversion into MeOH appears to be a promising route. Direct conversion of CO₂ into MeOH faces technical hurdles, however, that include slow kinetics and sensitivity to the water of conventional MeS catalysts and severe thermodynamic limitations. Most of the efforts to date have focused on the development of novel catalysts with improved kinetics over the conventional Cu—Zn MeS catalyst. However, such developments do not address the thermodynamic limitations associated with MeS and the correspondingly low single-pass conversions.

### SUMMARY

In at least one aspect, a post-combustion CO₂ capture and utilization (CCU) technology that converts the CO₂ into methanol (MeOH), a valuable chemical, thus providing a way to monetize the carbon captured to offset process costs. Methanol synthesis (MeS) has been discussed recently for application to CCU, but thermodynamic limitations make it difficult to convert in a single pass a large CO₂ fraction. Conventional catalysts show slow kinetics in converting CO₂-rich syngas (or pure CO₂) into MeOH. Our Group developed [1] a novel MeS process, employing a membrane contactor reactor (MCR) system that attains carbon conversions significantly higher than equilibrium. Our focus here is to process pure CO₂ streams by combining the MCR with a separate reactor, which converts the CO₂ into a syngas via the reverse water gas shift (RWGS) reaction. In this preliminary effort, the RWGS reactor (RWGSR) is assumed to reach equilibrium. Additional MeS kinetic rate data are generated validating experimentally the ability of the MeS-MCR to process as a feed the RWGSR exit stream. The performance of the combined (RWGSR/MeS-MCR) system is then simulated using a recently developed MeS-MCR model [2]. The findings are encouraging, and research is currently ongoing to experimentally validate the RWGSR/MeS-MCR system performance.

In another aspect, a novel direct CO₂ into MeOH conversion process that overcomes the limitations faced by current CO₂-based MeS processes is provided. The process is inspired by the two-step CAMERE design [22,23]. However, for the second stage, instead of a conventional PBR our process employs the MeS-MCR system recently developed by our team [1,2,27] that helps overcome the thermodynamic limitations associated with the MeS reaction, by removing MeOH and water in situ during the reaction, and which attains conversions nearing 90% or higher. The MeS-MCR concept also helps to overcome the other key challenge that MeS faces, which is accommodating the exothermicity of the reaction via a recirculating sweep solvent. Its modular character, furthermore, makes it ideal for distributed-type of applications, which is not always the case for the large-scale commercial MeS processes, which benefit from economy of scale [28], and do not always down-scale properly.

In another aspect, the application of this novel two-stage RWGSR/MeS-MCR system for processing pure CO₂ feeds is explored. In the systems studied, the composition of the exit stream from the RWGSR is simulated on the assumption that the reactor reaches equilibrium. Additional MeS kinetic rate data are generated, beyond those in our earlier efforts, focusing on validating experimentally the ability of the MeS-MCR to process as a feed the RWGSR exit stream. The performance of the combined (RWGSR/MeS-MCR) system is then simulated using a recently developed MeS-MCR model [2]. Research is currently ongoing in the group to experimentally study the performance of the integrated (RWGSR/MeS-MCR) system, and in future publications, we hope to validate the findings of this modeling effort with the results of these experimental studies.

In another aspect, a system for converting CO₂ to methanol is provided. The system includes a reverse water gas shift (“RWGS”) reactor configured to receive a first CO₂ stream and a hydrogen gas stream under a sufficient temperature and a sufficient pressure for an RWGS reaction to proceed. The RWGS reactor outputs an exit stream that includes CO. The system also includes a heat exchanger/condenser in fluid communication with the RWGS reactor configured to remove water from products of the RWGS reaction to form a dried exit stream that includes CO; and a membrane contactor reactor configured to receive a combination of hydrogen, CO₂, and the dried exit stream. The membrane contactor reactor is also configured to output a first output stream including methanol dissolved in a sweep liquid and a second output stream including gaseous H₂, gaseous CO, gaseous CO₂, and gaseous methanol. The membrane contactor reactor includes a tubular ceramic membrane having an interior and an exterior. The membrane contactor reactor also includes a packed-bed of methanol synthesis (“MeS”) catalysts surrounding the exterior of the tubular ceramic membrane. The tubular ceramic membrane is configured to allow the flow of the sweep liquid therethrough such that methanol formed in the packed-bed of MeS catalysts is transported through the tubular ceramic membrane and dissolves in the sweep liquid to form the first output stream. Characteristically, the interior of the tubular ceramic membrane defines a permeate-side and the packed-bed of MeS catalysts defines a reject-side.

In another aspect, a method for converting CO₂ to methanol using the system for converting CO₂ to methanol is provided. The method includes a step of providing CO, hydrogen, and CO₂, to a membrane contactor reactor. The membrane contactor reactor is configured to output a first output stream including methanol dissolved in a sweep liquid and a second output stream including gaseous H₂, gaseous CO, gaseous CO₂, and gaseous methanol. The membrane contactor reactor includes a tubular ceramic membrane having an interior and an exterior. The membrane contactor reactor further includes a packed-bed of methanol synthesis (“MeS”) catalysts surrounding the exterior of the tubular ceramic membrane. The tubular ceramic membrane is configured to allow flow of the sweep liquid therethrough such that methanol formed in the packed-bed of MeS catalysts is transported through the tubular ceramic membrane and dissolves in the sweep liquid to form the first output stream. The interior of the tubular ceramic membrane defines a permeate-side and the packed-bed of MeS catalysts defines a reject-side. The method also includes a step of separating methanol from the sweep liquid in the first output stream.

In another aspect, the methanol is separated from the sweep liquid by distillation.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1A. Schematic (i.e., a 2-Dimensional P&ID” of a combined RWGSR/MCR-MeS system.

FIG. 1B. Schematic of the MeS-MCR.

FIG. 2. Schematic of a variation the combined RWGSR/MCR-MeS system of FIG. 1A.

FIG. 3. CO₂ equilibrium conversion (left y-axis) of the RWGSR and the CF of the exit stream (right y-axis) versus the feed H₂/CO₂ molar ratio at different reactor exit temperatures.

FIGS. 4A, 4B, and 4C. MeS-PBR carbon conversion versus temperature and CF at different pressures A) P=20 bar, B) P=25 bar, C) P=30 bar. Other conditions SN=2 and W/F=20 g*hr/mol (red dots represent the experimental data, and the surfaces are calculated using the kinetics model presented in [2]).

FIGS. 5A, 5B, and 5C. MeS-PBR carbon conversion versus pressure and CF at different temperatures A) T=200° C., B) T=220° C., C) T=240° C. Other conditions SN=2 and W/F=20 g*hr/mol (red dots represent the experimental data, and the surfaces are calculated using the global rate expression model in [2]).

FIGS. 6A, 6B, 6C and 6D. RWGSR/MeS-MCR and RWGSR/MeS-PBR and calculated equilibrium carbon conversions versus CF (bottom x-axis) and RWGSR exit temperature (top x-axis) at different MCR temperatures: A) T=210° C., top left plot; B) T=220° C., top right plot; C) T=230° C., bottom left plot; D) T=240° C., bottom right plot. Other conditions, W/F=50 gr.hr/mol, SN=2, sweep liquid flow rate 6 cc/min, and P=30 bar.

FIGS. 7A, 7B, 7C, and 7D. RWGSR/MeS-MCR and RWGSR/MeS-PBR and calculated equilibrium carbon conversions versus CF at different pressures: A) P=20 bar, top left plot; B) P=30 bar, top right plot; C) P=35 bar, bottom left plot; D) P=40 bar, bottom right plot. Other conditions, W/F=50 gr.hr/mol, SN=2, sweep liquid flow rate 6 cc/min, and T=230° C.

FIGS. 8A, 8B, 8C, 8D, 8E, and 8F. RWGSR/MeS-MCR and RWGSR/MeS-PBR and calculated equilibrium carbon conversions versus W/F at different temperatures. Other conditions SN=2, sweep liquid flow rate 6 cc/min, and P=30 bar. CF=0.35 (left-side plots; A), C), and E)) and CF=0.7 (right-side plots; B), D), and F)).

FIGS. 9A, 9B, 9C, and 9D. RWGSR/MeS-MCR and RWGSR/MeS-PBR and calculated equilibrium carbon conversions versus sweep liquid flow rate at different W/F. Other conditions SN=2, T=230° C., and P=30 bar. CF=0.35 (left-side plots; a) and C)) and CF=0.7 (right-side plots; B) and D)).

FIGS. 10A, 10b, and 10C. RWGSR/MeS-MCR and RWGSR/MeS-PBR and calculated equilibrium carbon conversions versus CF for different (H₂/CO₂) feed molar ratios: a) H₂/CO₂₌₃, top left plot; b) H₂/CO₂=3.5, top right plot; c) H₂/CO₂₌₄, bottom plot. Other conditions, T=230° C., sweep liquid flow rate 6 cc/min, W/F=50 gr.hr/mol, and P=30 bar.

FIGS. 11A, 11B, and 11C. RWGSR/MeS-MCR and RWGSR/MeS-PBR and calculated equilibrium carbon conversions versus CF for different fractions of water removed from the RWGSR exit stream: a) complete removal, top left plot; b) 50% removal, top right plot; c) no removal, bottom plot. Other conditions, SN=2, T=230° C., sweep liquid flow rate 6 cc/min, W/F=50 gr.hr/mol, and P=30 bar.

FIGS. 12A, 12B, 12C, 12D, and 12E Process flow diagrams for the five different MeOH production technologies.

FIGS. 13A, 13B, AND 13C. Carbon conversion achieved by the RWGSR/MeS-MCR compared to that of a conventional PBR system as a function of A) mass of catalyst per molar feed flow rate (W/F), B) membrane surface area per mass of catalyst (A/W), and C) sweep liquid flow rate per mass of catalyst (F_{II}/W).

FIGS. 14A and 14B. (A) MSP comparison, and (B) cost breakdown for the five CO₂ to MeOH processes.

FIGS. 15A and 15B. A) Energy efficiency comparison, and B) energy consumption breakdown comparison among the five CO2-to-MeOH processes.

FIGS. 16A, 16B, 16C, and 16D. MSP comparison among RWGSR/MeS-MCR, CAMERE, and Direct CO₂ to MeOH technologies as a function of a) CO₂ cost, b) H₂ cost, c) project capacity considering base case scenario, and d) project capacity considering optimistic scenario.

FIGS. 17A, 17B, and 17C. Comparison between the DPBP of the RWGSR/MeS-MCR and the two other catalytic technologies under an optimistic scenario considering A) a MrOH selling price of $400/ton, B) a MeOH selling price of $600/ton as well as C) the NPV of RWGSR/MeS-MCR technology compared to those of the other two catalytic technologies under a 20× capacity scenario and a MeOH selling price of $600/ton.

FIGS. 18A and 18B. MSP of the RWGSR/MeS-MCR technology for various membrane costs as a function of A) the sweep solvent cost, and B) membrane lifetime.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

Throughout this application, where publications are referenced, the disclosures of these publications in their entireties are hereby incorporated by reference into this application to more fully describe the state of the art to which this invention pertains.

### ABBREVIATIONS

“comp” means compressor.
“E” means heat exchanger.
“MeS” means methanol synthesis.
“MCR” means membrane contactor reactor.
“MFC” means mass flow controller.
“MX” means mixer.
“P” means pump.
“P&ID” means a piping and instrumentations diagram.
“PBR” means packed-bed reactor.
“PEM” means proton exchange membrane.
“RWGSR” means reverse water gas shift reactor.
“WGS” means water gas shift.

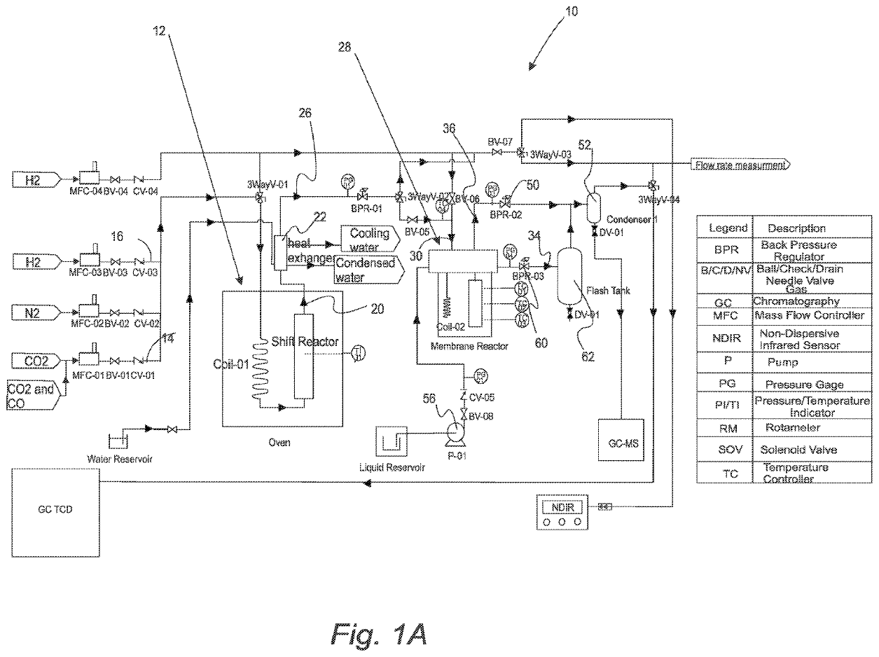

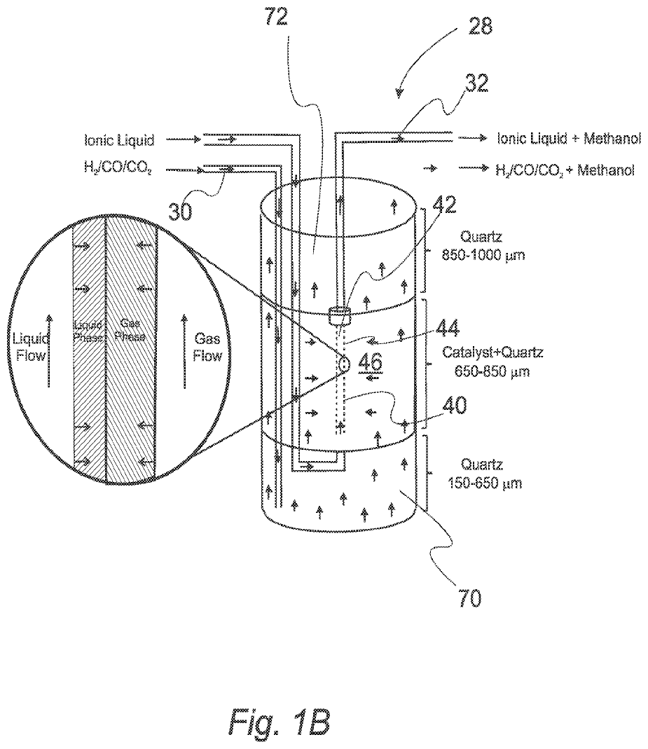

Referring to FIG. 1A, a schematic of a system for converting CO₂ to methanol is provided. System 10 includes reverse water gas shift (“RWGS”) reactor 12 configured to receive a first CO₂ stream 14 and a hydrogen gas (i.e., H₂) stream 16 under a sufficient temperature and pressure for an RWGS reaction to proceed. In a refinement, the hydrogen gas is produced by a PEM electrolyzer (e.g., with a cell voltage=2.4 V and current density=1.5 A/cm²). In a further refinement, the hydrogen gas stream is mixed with the first CO₂ stream with a (H₂/CO₂ molar ratio of 1 to 5 or 2:1 to 4:1. The first CO₂ stream 14 and the hydrogen gas are received under a sufficient temperature and a sufficient pressure for an RWGS reaction to proceed. In a refinement, the sufficient temperature is from about 500° C. to about 700° C. In a further refinement, the sufficient pressure is from about 0.8 bar to about 3 bar (optimally about 1 bar).

Still referring to FIG. 1A, RWGS reactor 12 outputs an exit stream 20 that includes CO (and typically water and H₂). In a refinement, the hydrogen content of the exit stream can be adjusted via a separate H₂ line. A heat exchanger/condenser 22 is in fluid communication with the RWGS reactor and configured to remove water from products of the RWGS reaction to form a dried exit stream 26 that includes CO. Membrane contactor reactor 28 (i.e., the MeS-MCR) is configured to receive a combined stream 30 of hydrogen (H₂), CO₂, and the dried exit stream 26. Membrane contactor reactor 28 is also configured to output a first output stream 34 including methanol dissolved in a sweep liquid and a second output stream 36 including gaseous H₂, gaseous CO, gaseous CO₂, and gaseous methanol. Advantageously, at least a portion of the CO₂ in first CO₂ stream 14 and the CO₂ provided to the membrane contactor reactor 28 can be derived from flue gas. In a refinement, membrane contactor reactor 28 operates at a temperature from about 150 to 250° C. and a pressure from about 10 to 50 bar.

Referring to FIGS. 1A and 1B, membrane contactor reactor 28 includes a tubular ceramic membrane 40 having an interior 42 and an exterior 44. Membrane contactor reactor 28 also includes a packed-bed of methanol synthesis (“MeS”) catalysts 46 surrounding the exterior of the tubular ceramic membrane 40. Tubular ceramic membrane 40 is configured to allow flow of the sweep liquid therethrough such that methanol formed in the packed-bed of MeS catalysts 46 is transported through the tubular ceramic membrane and dissolves in the sweep liquid to form the first output stream 34. Characteristically, the interior 42 of the tubular ceramic membrane defines a permeate-side and the packed-bed of MeS catalysts 46 defines a reject-side.

In a refinement, the MeS catalysts include a Cu-based MeS catalyst. In a further refinement, the MeS catalyst is a copper-zinc catalyst that includes copper-zinc. For example, the MeS catalysts include zinc oxide and alumina as a support with copper as an active catalytic component.

Referring to FIG. 1A, a reject-side back pressure regulator 50 to control the pressure of the reject-side. System 10 can also include a reject-side condenser 52 at an outlet of the reject-side to ensure that complete condensation takes place for MeOH, H₂O, and other potential gas phase by-products exiting the membrane contactor reactor.

Still referring to FIG. 1A, system 10 can also include HPLC pump 56 that injects the sweep liquid is injected into the permeate-side. In a refinement, the sweep liquid is a petroleum-derived solvent or an ionic solvent.

Still referring to FIG. 1A, system 10 also includes permeate-side back pressure regulator 60 in fluid communication with the membrane contactor reactor 28. In a refinement, system 10 also includes a liquid condenser 62 in fluid communication with the permeate-side back pressure regulator 28 and operates at atmospheric pressure and room temperature to separate any such gas components from the sweep liquid. In a further refinement, a gas stream from the liquid condenser 62 is recombined with the gas stream exiting the reject-side back pressure regulator 50 with a resulting total gas stream then being fed into the reject-side condenser 52.

Referring to FIG. 1B, contactor reactor 28 can include one or more packed beds 70 and 72 below (i.e., downstream) and/or above (upstream) packed-bed of MeS catalysts 46. In a refinement, these additional packed beds include chemically inert material such as quarts or sand. Additional details for the membrane contactor reactor 28 are set forth below.

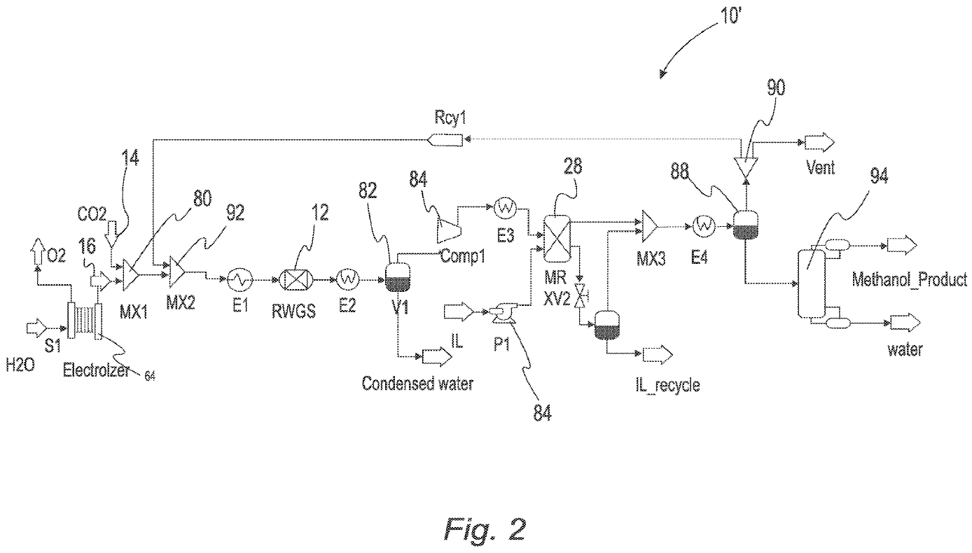

Referring to FIG. 2, a schematic of a variation of the system for converting CO₂ to methanol of FIG. 1A is provided. System 10′ includes reverse water gas shift (“RWGS”) reactor 12 configured to receive a first CO₂ stream 14 and a hydrogen gas (i.e., H₂) stream 16 under a sufficient temperature and pressure for an RWGS reaction to proceed. In a refinement, the hydrogen gas is produced by a PEM electrolyzer 64 (e.g. with a cell voltage=2.4 V and current density=1.5 A/cm²). In a further refinement, the hydrogen gas stream is mixed at mixer 80 with the first CO₂ stream with a (H₂/CO₂ molar ratio of 1 to 5 or 2:1 to 4:1. The first CO₂ stream 14 and the hydrogen gas stream 16 are received into RWGS reactor 12 under a sufficient temperature and a sufficient pressure for an RWGS reaction to proceed. In a refinement, the sufficient temperature is from about 500° C. to about 700° C. In a further refinement, the sufficient pressure is from about 0.8 bar to about 3 bar (optimally about 1 bar).

Still referring to FIG. 2, in the next step, the water produced by the RWGS reaction is removed from the gas steam exiting the RWGSR reactor 12 using a condenser 82, and the dried syngas is then compressed by the compressor 84 and fed to the shell side of a MeS-MCR 28 where it is converted into MeOH. Details of the MeS-MCR 28 are the same as set forth above. In a refinement, membrane contactor reactor 28 operates at a temperature from about 150 to 250° C. and a pressure from about 10 to 50 bar. Simultaneously, a solvent is pumped via pump 86 through the tube-side (membranes) of the reactor 28, acting both as a coolant and as a sweep stream to remove in situ the reaction products. Removing the MeOH and water produced by the catalytic reaction from the reactor shell-side and transporting them through the membrane into the reactor tube-side, where they are dissolved in the sweep solvent, helps to overcome the thermodynamic limitations associated with the MeS reaction, and to attain conversions of −80% or higher. In the next step, the unreacted syngas is separated from the MeOH/water mixture in a condenser 88 and recycled back to the RWGSR through splitter 90 to mixer 92, and the sweep solvent is regenerated. Finally, the MeOH/water mixture is fed into a distillation column 94 to produce purified MeOH as the final product.

The following examples illustrate the various embodiments of the present invention. Those skilled in the art will recognize many variations that are within the spirit of the present invention and the scope of the claims.

### 1. Carbon Dioxide Capture From Flue Gas

### 1.1. Experimental Section

Technical details regarding the experimental MeS-MCR set-up, the catalyst and membrane used, membrane modification, gas, and liquid measurement methods, system and data analysis are described in previous papers by our group [1,27]. We utilize a commercial Cu-based MeS catalyst (MK-121, purchased from Haldor-Topsoe), with properties reported in [1,27]. We also employ a mesoporous multilayer ceramic membrane from Media and Process Technology, Inc. (M&PT) of Pittsburgh, PA. As a sweep fluid, we employ two different liquids, a petroleum-derived solvent tetraethylene glycol dimethyl ether (TGDE) [1] and an ionic liquid, 1-ethyl-3-methylimidazolium tetrafluoroborate ([EMIM][BF₄]) [27]. In the simulations reported here, we utilize the TGDE as the solvent, as the MeS-MCR model we utilize has been previously validated with data employing this solvent.

A schematic of the lab-scale system is shown in FIG. 1, and details can be found in recent reports [29]. It basically consists of the gas delivery system, encompassing the required gas cylinders, each connected to an individual mass flow controller (MFC), the high-pressure RWGSR, followed by the MeS-MCR and appropriate analytical hardware for the analysis of the feed and exit gas streams of the RWGSR, and the gas and liquid streams for the MeS-MCR. The exit stream from the RWGSR, prior to being fed into the MeS-MCR, passes through a heat exchanger/condenser to remove the water product of the RWGS reaction. The H₂ content of that stream can be adjusted via a separate H₂ line, if so desired.

FIG. 2 shows a schematic of the MCR itself. The reactor is divided into two zones by the tubular ceramic membrane: The shell-side (reject-side) and the tube-side (permeate-side). The shell-side contains a packed-bed of catalysts and the MeS reaction occurs in this zone. The MeOH produced is then transported through the membrane and is dissolved into the sweep liquid (TGDE or [EMIM][BF4]) that flows in the tube-side. A back-pressure regulator (BPR) is used to control the pressure of the MR shell-side. A condenser is installed at the outlet of the shell-side to ensure that complete condensation takes place of the MeOH, H₂O, and other potential by-products in the gas phase exiting the reactor.

The sweep liquid is injected into the membrane permeate-side using a HPLC pump, with a BPR (installed in the exit line) being employed for controlling the pressure. Due to the potential of gases being dissolved in the sweep liquid, it is directed after the BPR into a condenser/separator operating at atmospheric pressure and room temperature, to separate any such gas components from the sweep liquid. For the purpose of properly closing mass balances, the gas stream from the liquid condenser is recombined with the gas stream exiting the BPR in the MCR shell-side, the resulting total gas stream then being fed into the shell-side condenser. The same experimental set-up is used for performing the MeS-PBR experiments that are reported here; this is accomplished by closing both the inlet and outlet lines in the tube-side of the membrane.

### 1.2. RESULTS AND DISCUSSION

A Fe—Cu—Cs/Al₂O₃ supported RWGS catalyst which was recently reported by Pastor-Perez et al. [30,31] was employed to show highly selective and stable RWGS performance. The focus of our efforts is to extend the range of experimental conditions studied to higher pressures that are, potentially, more relevant for the proposed MeS-based CCU process, and to develop a data-validated global rate expression that will allow further process development and scale-up: The study of Pastor-Perez et al. [30,31] was carried out at atmospheric pressure conditions, and they did not report a reaction rate expression.

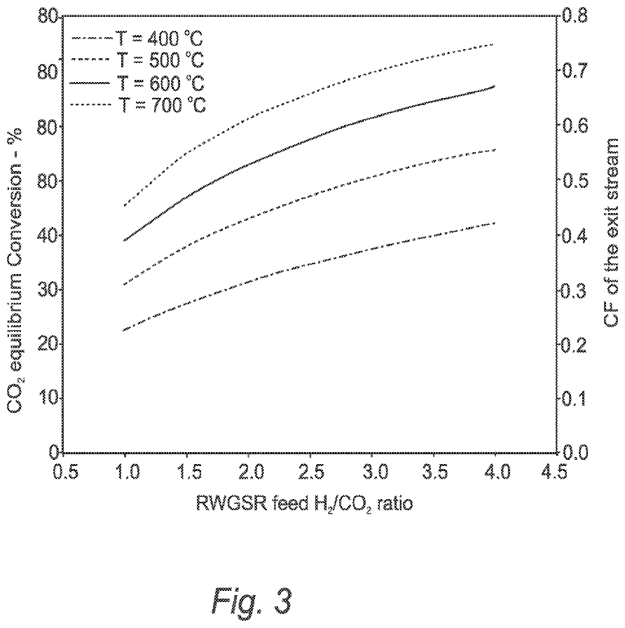

For the present study, we have assumed that the quantity of catalyst used in the RWGSR is sufficiently high so that equilibrium conditions are attained at the reactor's exit for all conditions studied. We proceeded then to calculate these exit compositions, which were subsequently utilized as feeds in the MeS kinetic experiments. In the simulations, we utilized the equilibrium constant reported by Moe [32]. We also assumed, in this preliminary project phase, that the feed to the RWGSR consists of a CO₂/H₂ gas mixture, with the CO₂ stream being separated and captured from the flue gas of a power plant via a post-combustion step (later in this research, once our studies with pure CO₂ feeds are completed, we plan to also investigate the direct utilization of flue gas). In FIG. 3, we plot (left y-axis) the equilibrium reactor conversion versus the (H₂/CO₂) molar ratio in the RWGSR feed. Since the RWGS reaction is endothermic, as expected, the CO₂ conversion increases with temperature. The CO₂ conversion also increases as the (H₂/CO₂) ratio increases.

In the MeS experiments, in addition to the reactor temperature (T), pressure (P), and inlet molar flow rate (specifically, the catalyst weight to molar flow rate ratio−W/F), the feed composition is also a key consideration. Traditionally, in MeS reactor design the syngas feed composition is characterized by two quantities: The carbon factor (CF=mol CO/(mol CO+mol CO₂)), and the feed stoichiometric number (SN=(mol H₂—mol CO₂)/(mol CO+mol CO₂)). In the proposed process, the outlet flow from the RWGSR constitutes the feed to the MeS reactor, so the SN remains invariant among the two reactors (SN=2 corresponds to the stoichiometric ratio of H₂/CO₂=3−note, though, that the present experimental set-up shown in FIG. 1 allows one to add more H₂ to the MCR feed, if so desired). The CF depends on the conversion of the RWGSR, and it is plotted for different temperatures as a function of the (H₂/CO₂) molar ratio in the feed of the RWGSR in FIG. 3 (right y-axis). The CF increases with increasing temperature and with the (H₂/CO₂) molar ratio. Typically, conventional MeS reactors operate with a CF in the range of 0.6-0.7, which is also the range of CF values in which we previously operated the MeS-MCR [1,27]. For a feed SN=2 (corresponding to a stoichiometric H₂/CO₂ feed molar ratio equal to 3) to attain such CF values, the operating RWGSR temperatures should be in the range of 600° C.-700° C., which is in line with the operating temperatures of the original CAMERE process. Operating the RWGSR at such high CO₂ conversion levels is costly (the RWGS reaction is strongly endothermic), so a key objective of this research was to investigate the performance of the MeS—MCR system when operating with feeds with significantly lower CF values.

### 1.2.1 Packed-Bed Reactor Experimental Results

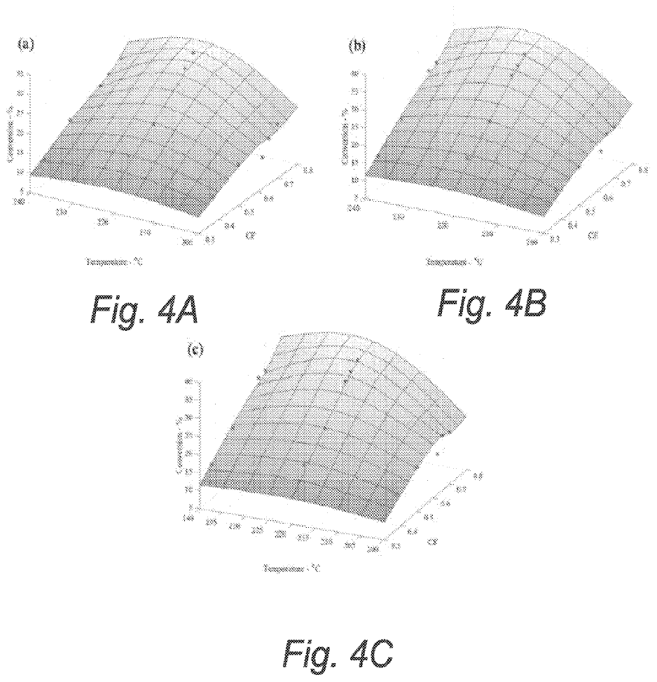

FIG. 4 shows the experimental carbon (CO+CO₂) conversions measured in the MeS-MCR set-up, in its operating mode as a PBR (with the inlet and outlet of the membrane being kept closed and with no liquid sweep flowing). In these experiments, we used a simulated RWGSR exit stream, which, in line with the original CAMERE process, contains only CO/CO₂/H₂.

This assumes that the heat exchanger/condenser downstream of the RWGSR (see FIG. 1) will remove all the H₂O from the RWGSR exit stream (during the operation of the combined RWGSR/MeS-MCR experimental system, we expect some small concentration of H₂O to remain in the MCR feed stream since, for experimental convenience, we use tap water as the coolant—if that turns out to have a significant impact on performance, we will consider using a different coolant). The reason for doing so is that preliminary mathematical simulations show that removing the water from the feed stream has a very positive impact on MeS-PBR conversion; this is true for the MeS-MCR performance as well, though less so since a key characteristic of the latter reactor is to remove in situ the water that is produced during MeS. Removing the water from the exit stream of the RWGSR confers additional process complexity, however, since one must cool the stream first and then reheat it to the temperature of the MeS step. In our future process design and technical and economic analysis (TEA) efforts, we will investigate, therefore, whether only partial removal of the water content of the RWGSR exit stream is a more optimal condition for the operation of the combined system.

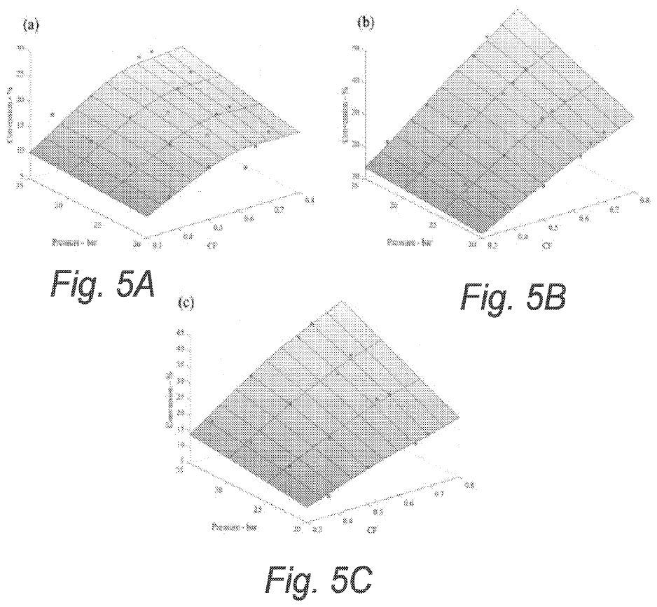

In the experiments presented here (the experimental uncertainty of the carbon conversions shown in FIGS. 4 and 5 is less than 2%, and we have observed no catalyst deactivation), we utilize SN=2, corresponding to a (H₂/CO₂) feed molar ratio for the RWGSR equal to 3, as noted above. Using (H₂/CO₂) ratios greater than 3 will increase the CF of the exit stream from the RWGSR (that serves as feed for the MeS reactor) as FIG. 3 shows, but this also requires a greater quantity of H₂ to be used, which for CCS processes implies a higher energetic penalty per ton of CO₂ avoided. One observes from FIG. 4, that the CF of the feed for the MeS-PBR has a significant impact on conversion, with the conversion (at constant reactor temperature) increasing substantially as CF increases. The impact of temperature (at constant CF) is typical of that of exothermic reactions, with conversion first increasing as the temperature increases and then decreasing again, as the exit conditions begin tracking the equilibrium conversion line, since for exothermic reactions like MeS the equilibrium conversion decreases with temperature. FIG. 5 shows the carbon conversions in the MeS-PBR as a function of CF and pressure. Pressure, as expected, has a positive impact on reactor conversion.

The surfaces shown in FIGS. 4 and 5 represent the results of simulations of the PBR utilizing the MeS global reaction rate expressions previously reported [2]. The model was analyzed with an in-house computer program. There is good agreement between the experimental data and the model results for a broad range of temperatures, pressures, and feed compositions, with the standard deviation between the experimental and the modeled carbon conversions shown in FIGS. 4 and 5 being less than 2.19%. This further validates these global rate expressions. The same kinetics are further utilized here to simulate the behavior of the combined RWGSR/MeS-MCR system

### 1.2.2 Modeling the Behavior of the RWGSR/MeS-MCR System

Next, we simulate the behavior of the integrated lab-scale RWGSR/MeS-MCR system employing the MeS-MCR model described in our recent paper [2]. The MeS-MCR employs a mesoporous alumina membrane whose properties are reported in that publication. The MeS-MCR model employs the Dusty Gas Model (DGM) to describe gas transport through the 3-layer membrane structure, the Wilke-Chang formulation to describe transport through the liquid-filled part of the membrane structure, and the SRK equation of state (EOS) model to describe the gas solubility in the liquid. TGDE is utilized as the sweep solvent. As noted above, in the simulations we assume that the RWGSR operates under equilibrium conditions and that the exit stream from that reactor, after its water content is removed, serves as the feed to the MeS-MCR in the second stage of the integrated system.

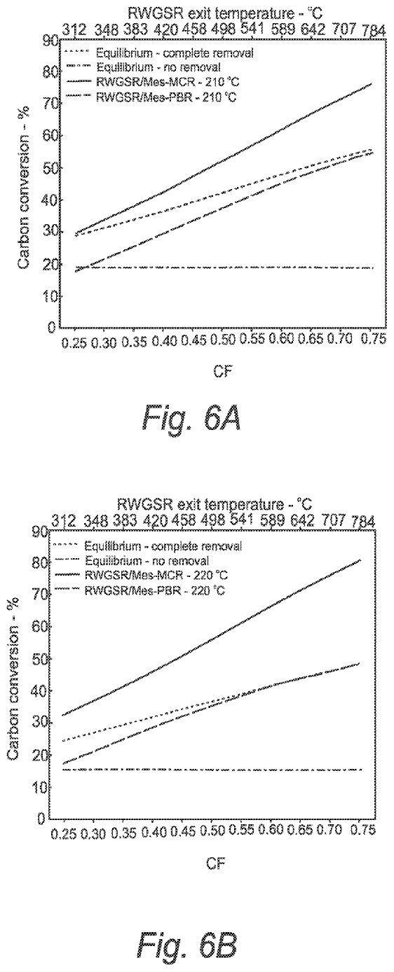

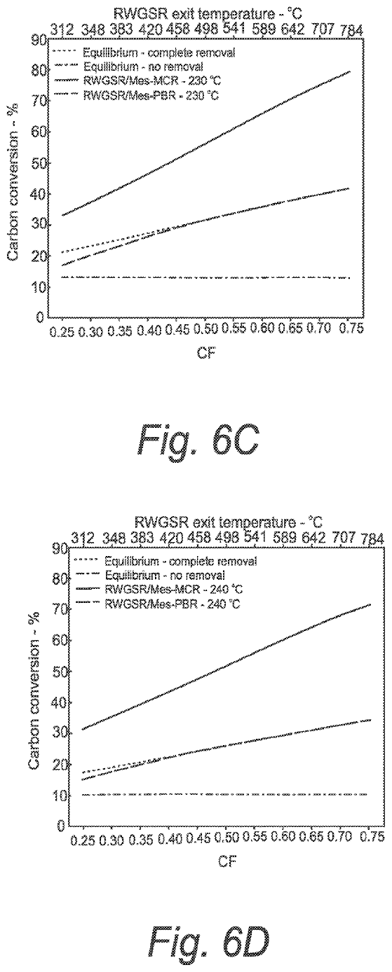

FIG. 6 shows the total carbon conversion of the integrated RWGSR/MeS-MCR system vs. CF (bottom X-axis) or equivalently the corresponding exit temperature from the RWGSR (top X-axis). In this example, we keep the ratio of the weight of the MeS catalyst (W) to the molar feed flow rate of CO₂/H₂ (F) into the reactor constant and equal to 50 gr.hr/mol. The pressure for the MeS-MCR is kept at 30 bar, and we investigate four different temperatures in the range of 200° C.-240° C.

The feed into the RWGSR is assumed to consist of H₂ and CO₂ with a (H₂/CO₂) molar ratio equal to 3 (SN=2). We assume that the MeS involves the following two reactions, with global reaction rates reported in our previous publication [2], and included in the Supplementary Materials section.

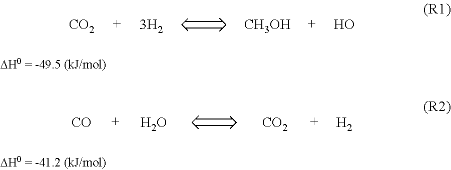

In addition to the carbon conversion for the RWGSR/MeS-MCR, reported in FIG. 6, we also plot the conversion of the RWGSR/MeS-PBR. The plots also include two different calculated equilibrium conversions. The first total carbon equilibrium conversion is for the case where the exit stream from the RWGSR is fed into the MCR without removing the water it contains. The second equilibrium conversion is for the case where the water is removed from the exit stream from the RWGSR, with the dry stream then being fed into the MCR stage.

As FIG. 6 shows, the carbon conversion for both the RWGSR/MeS-MCR and RWGSR/MeS-PBR increases as CF (and the corresponding exit temperature of the RWGSR) increases, which validates the advantage of the proposed two-stage, integrated CAMERE-type process. The conversion of the RWGSR/MeS-MCR exceeds the conversion of the RWGSR/MeS-PBR for all conditions studied. The equilibrium conversion for the case where the water is not removed from the exit stream of the RWGSR does not depend on the CF, as expected, and it is quite low. The equilibrium conversion for the case where the water is removed from the exit stream of the RWGSR before being fed into the MeS-MCR, on the other hand, increases as the CF increases. Both conversions decrease as the MCR temperature increases, as expected, since the MeS reaction is exothermic. The conversion of the RWGSR/MeS-MCR system is significantly higher than both equilibrium conversions. The conversion of the RWGSR/MeS-PBR is higher than the equilibrium conversion without interstage water removal but stays below the equilibrium conversion for the case with water removal, approaching such conversion for the higher CF values.

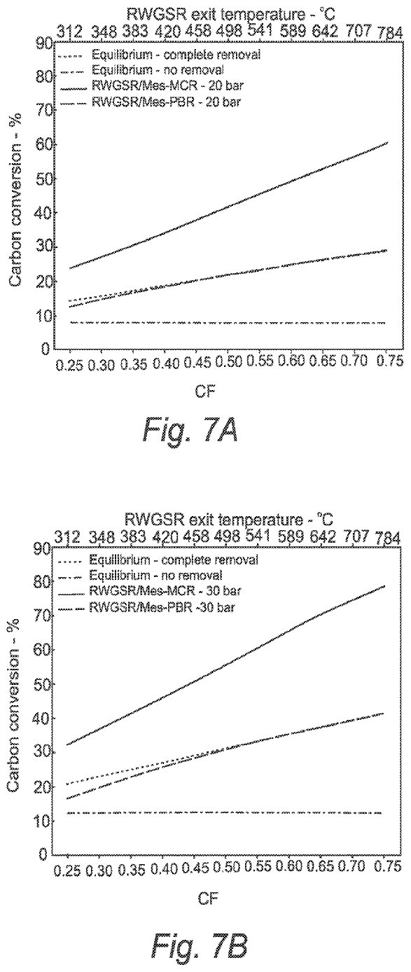

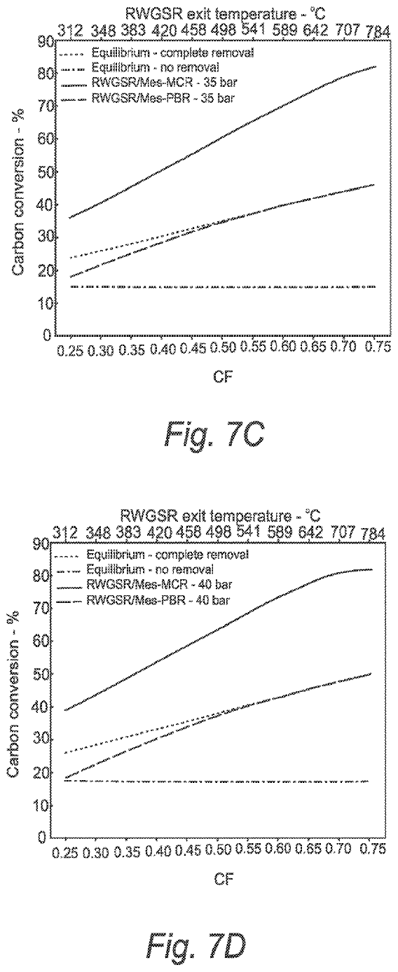

FIG. 7 shows the carbon conversion of the RWGSR/MeS-MCR, the RWGSR/MeS-PBR and the two different equilibrium conversions vs. CF (bottom X-axis) or the corresponding exit temperature from the RWGSR (top X-axis). The feed into the RWGSR is again assumed to consist of H₂ and CO₂ with a (H₂/CO₂) molar ratio equal to 3 (SN=2). We also keep the W/F=50 gr.hr/mol. The temperature for the MeS-MCR is kept constant at 230° C., and we investigate four different pressures in the range of 20 bar-40 bar.

Again, the positive impact of increasing CF (i.e., the RWGSR exit reactor temperature) is clear from this Figure. The RWGSR/MeS-MCR, once more, shows significantly improved conversions over the RWGSR/MeS-PBR. In fact, the conversion of the RWGSR/MeS-MCR with a certain CF exceeds the conversion of the RWGSR/MeS-PBR with a much higher CF value. The impact of pressure is as expected, with increasing pressures favorably impacting the conversion for both reactors. The RWGSR/MeS-MCR conversion is again significantly higher than the equilibrium conversions (both with and without interstage water removal). The conversion for the RWGSR/MeS-PBR, on the other hand, though higher than the equilibrium conversion without water removal, always stays below the equilibrium conversion with water removal, only approaching it for the higher CF values.

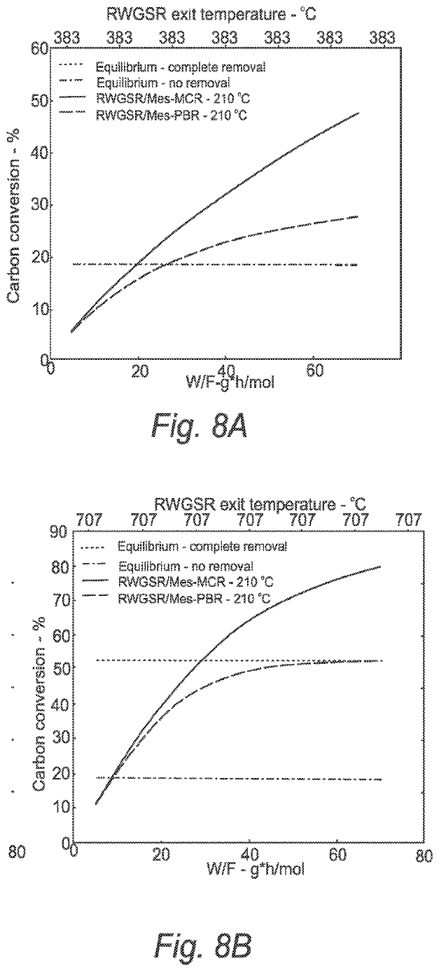

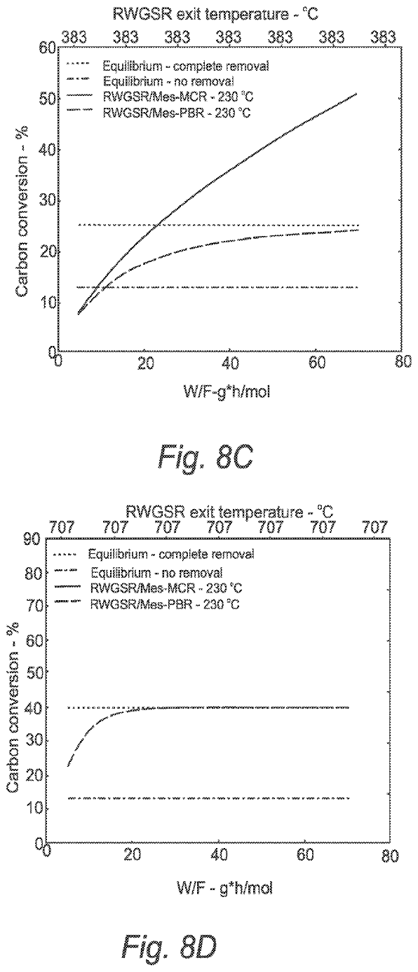

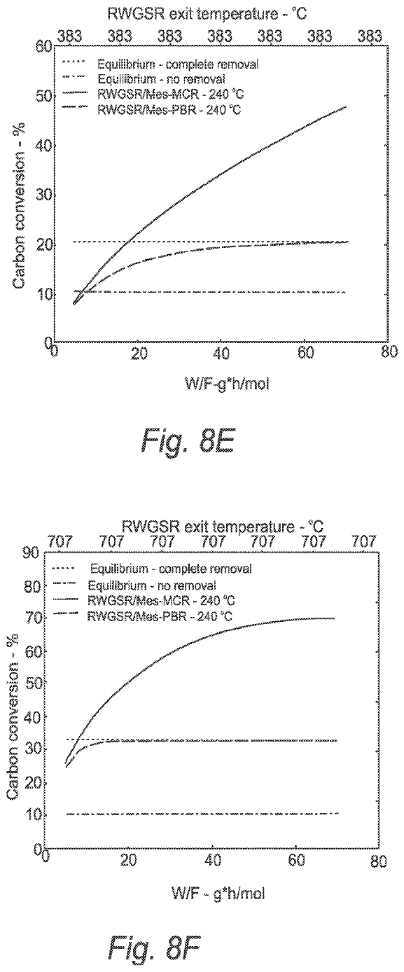

In FIG. 8, we report the carbon conversions of the RWGSR/MeS-MCR, the RWGSR/MeS-PBR and the two equilibrium conversions (with and without water removal) vs. W/F. The left-side plot is for CF=0.35 and the right-side plot is for a CF=0.7. In each plot, we show the results for three different temperatures. As W/F increases, the conversions for both reactors increase. For the RWGSR/MeS-PBR, the conversion at some value of W/F crosses the equilibrium value with no interstage water removal, but stays below the equilibrium conversions with interstage water removal, asymptotically approaching such equilibrium values for higher values of W/F. In contrast, the RWGSR/MeS-MCR conversion continues to increase finally exceeding the equilibrium conversion with water removal. The W/F where such crossovers in conversion take place decreases with increasing temperature. Combined with the fact that for the exothermic MeS reaction equilibrium conversion decreases with temperature, this then generates a very interesting and complex reactor behavior.

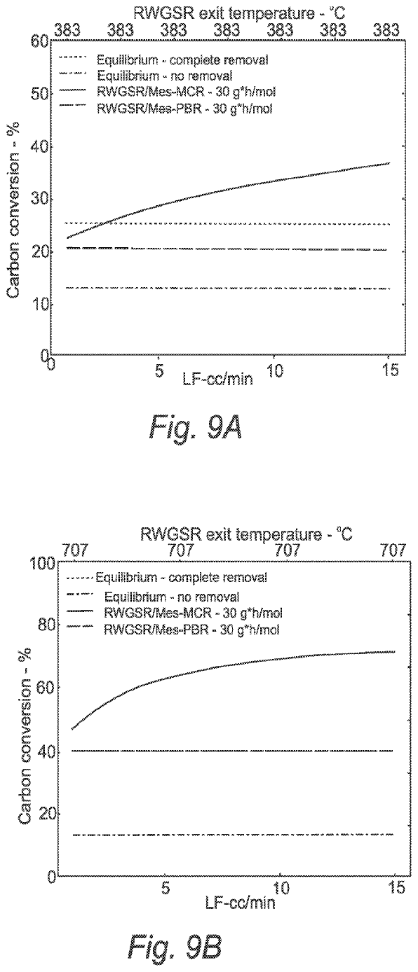

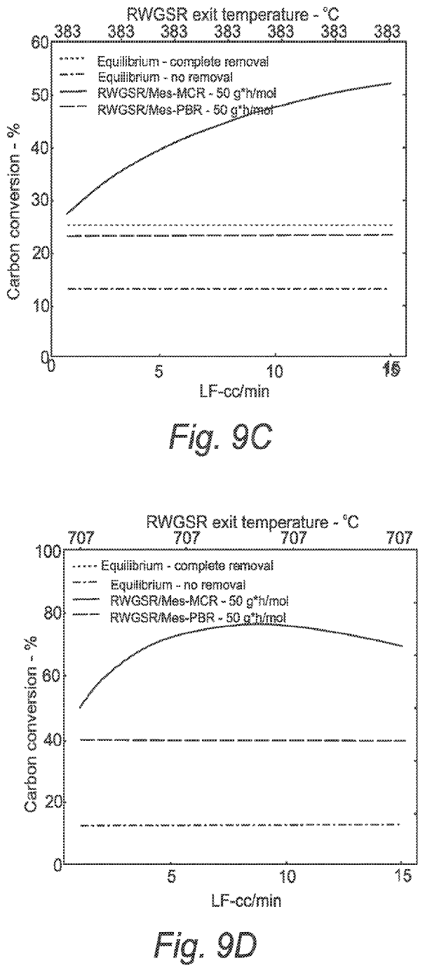

In FIG. 9, we plot the carbon conversions of the RWGSR/MeS-MCR, the RWGSR/MeS-PBR and the equilibrium conversions vs. the sweep liquid flow rate (the RWGSR/MeS-PBR conversion and the equilibrium conversions do not depend on the liquid flow rate and are, thus, shown as horizontal straight limes in these plots).

The left-side plot is for CF=0.35 and the right-side plot is for a CF=0.7. In each plot we show the results for two different W/F. As the sweep liquid flow rate increases, the conversions for both reactors increase, eventually exceeding the equilibrium conversions. However, for the higher W/F and CF cases, from a value of the liquid flow rate and beyond the conversion begins to decrease as a result of reactant dissolution in the sweep liquid phase.

Though such reactant dissolution has a negative impact on conversion, as shown in FIG. 9, it does not represent a further process impediment, however, since when the sweep liquid is depressurized these gases get desorbed, and in our own experimental set-up, as noted in Sect. 1.2, they are rejoined with the gas phase exiting the shell-side. Further, our recent experimental solubility studies [33] employing the ionic liquid as a solvent indicate that the syngas permanent gas components show no solubility in the IL at the typical MeS high temperature and pressure conditions, so reactant loss is no longer a concern.

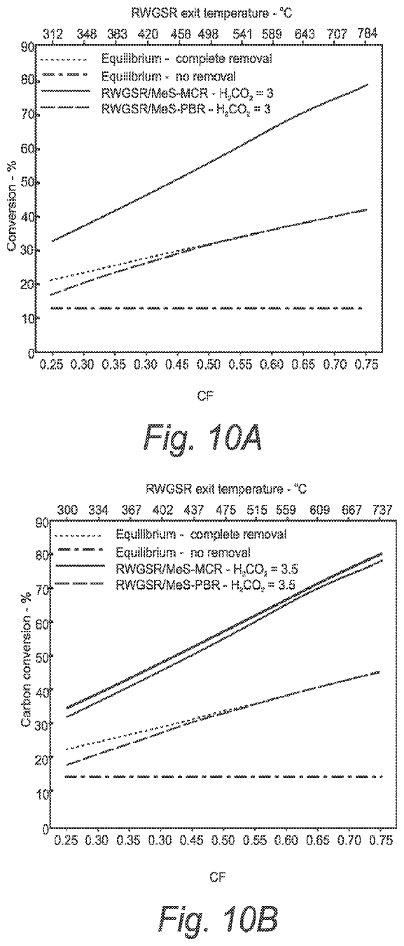

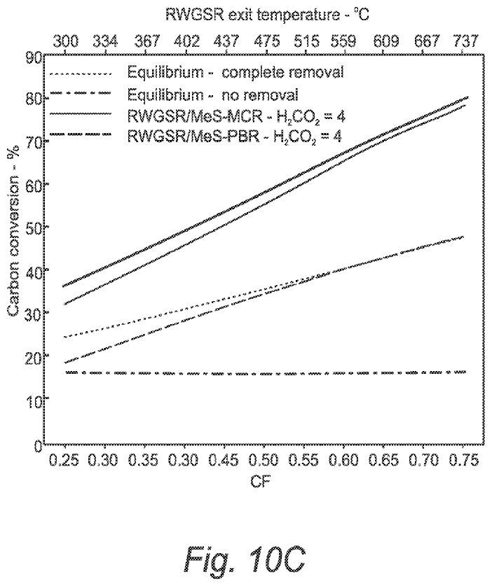

FIG. 10 investigates the impact of varying the (H₂/CO₂) molar flow ratio on system performance. The Figure shows the carbon conversion of the RWGSR/MeS-MCR and the RWGSR/MeS-PBR and the equilibrium conversions vs. CF.

Here, we keep the W/F=50 gr.hr/mol, the MeS-MCR temperature equal to 230° C., and the pressure equal to 30 bar. We investigate three different (H₂/CO₂) molar flow ratios: 3 (SN=2), left upper plot, 3.5 (SN=2.5) right upper plot, and 4 (SN=3) bottom plot. There is beneficial impact of increasing the (H₂/CO₂) molar flow ratio on reactor performance more so, however, for the RWGSR/MeS-PBR rather than the RWGSR/MeS-MCR case. The downside of using a higher (H₂/CO₂) is the need for providing additional H₂, though for the kind of high conversions attained in the RWGSR/MeS-MCR, recycling of the unreacted hydrogen with no further purification may become a viable option.

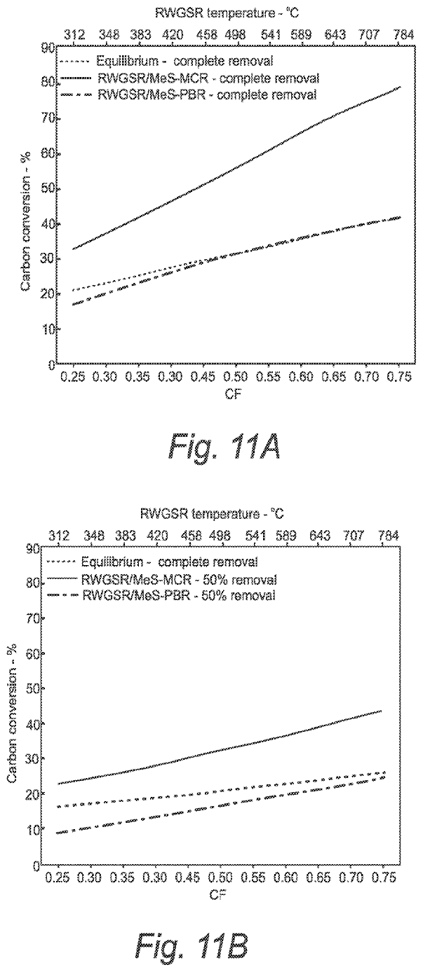

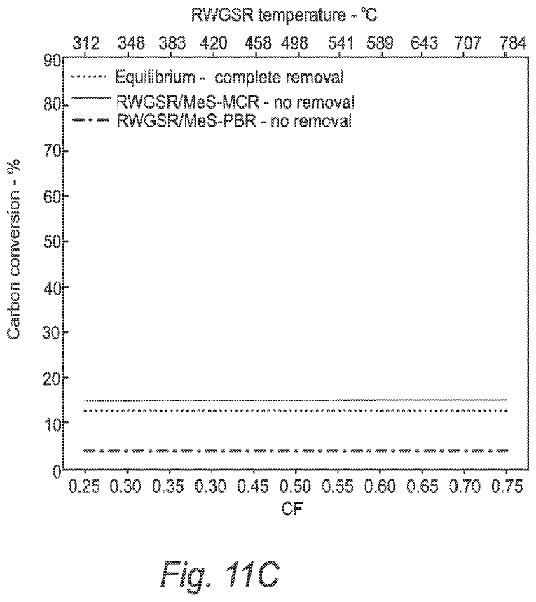

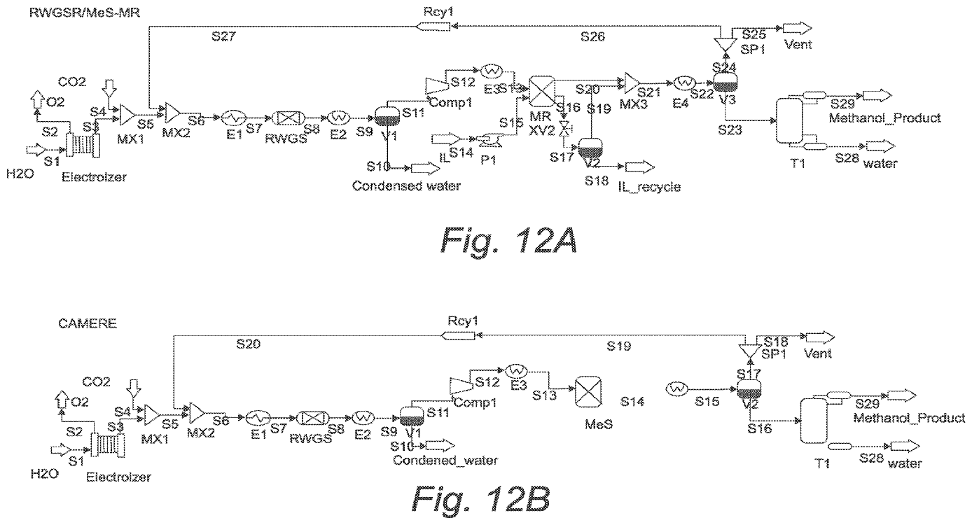

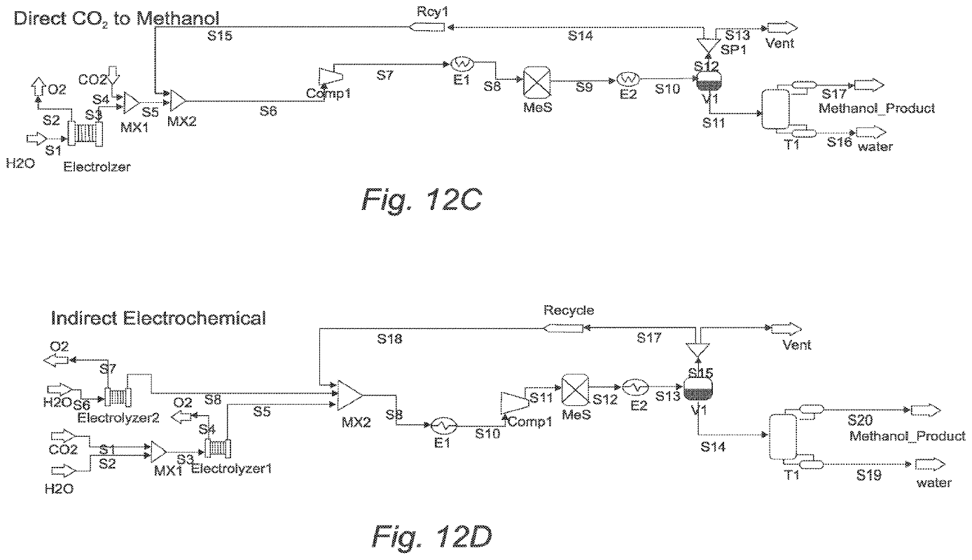

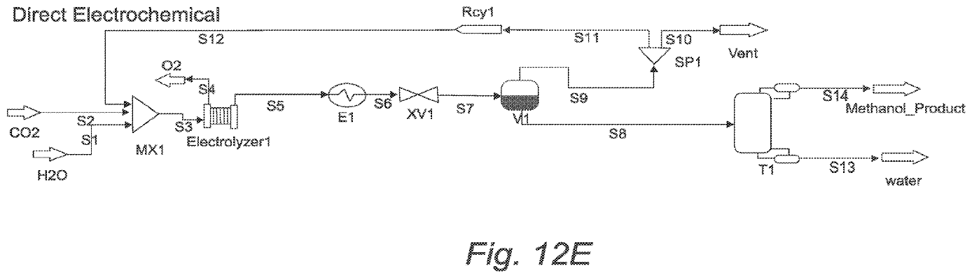

Finally, FIG. 11 shows the positive impact that removing the water from the exit stream from the RWGSR has on overall system performance. In this Figure, we plot the carbon conversion of the RWGSR/MeS-MCR, the RWGSR/MeS-PBR and the equilibrium conversion vs. CF (and exit temperature from the RWGSR). We study three different cases: Complete H₂O removal (base case), 0% H₂O removal and 50% water removal. All other conditions among the three cases remain the same (W/F=50 gr.hr/mol, SN=2, sweep liquid flow rate 6 cc/min, T=230° C., P=30 bar). The negative impact on reactor performance that the presence of H₂O in the feed of the 2nd stage MeS-MCR or MeS-PBR has on performance is quite clear. Water affects the conversion of the reactor in two different ways: It inhibits the MeS reaction kinetics and decreases the reactor residence time, the first effect being significantly more impactful. The effect of water is much more substantial on the performance of the RWGSR/MeS-PBR than on the RWGSR/MeS-MCR. The reason for that is much of the water produced in the latter reactor is removed through the membrane. This represents then an added advantage of the RWGSR/MeS-MCR over its more conventional counterpart.

### 1.3. CONCLUSIONS

A novel reactive separations technology (termed the RWGSR/MeS—MCR process) for the utilization of waste CO₂ streams is disclosed. This is a two-stage process that combines a reactor that converts waste CO₂ into a syngas mixture that can subsequently be processed in a membrane contactor reactor with high efficiency to produce valuable liquid products. This technology attains carbon conversion efficiencies in excess of 85%, significantly higher than the equilibrium values and those attained by more conventional reactors. In contrast with other past efforts, the membrane utilized in this work is an “off-the-shelf” commercial γ-alumina that serves as an interface contactor in between the MeS environment in the shell-side and a sweep liquid solvent flow in the membrane permeate-side. The MeS products (MeOH and H₂O) have high solubility in the sweep liquids but the permanent gases like H₂ and CO do not. Removing in situ the products generated, allows the reactor conversion to reach beyond equilibrium.

In the effort, the focus was on the performance of the MeS—MCR component of the integrated RWGSR/MeS-MCR process. For that, we utilized as feeds to the MeS-MCR unit simulated syngas mixtures with compositions that represent the exit stream compositions of the RWGSR operating under equilibrium conditions. In our research, we validated the ability of the integrated RWGSR/MeS-MCR system to process and efficiently convert pure CO₂ streams into MeOH. Higher conversions in the RWGSR 1st stage (greater CF values), larger (H₂/CO₂) molar feed ratios, and the removal of water from the exit stream of the RWGSR all favorably impact process performance. The findings of this preliminary investigation are encouraging, indicating the advantages offered by the combined RWGSR/MeS-MCR system over the conventional MeS-PBR as well as the stand-alone MeS-MCR systems. Research is currently ongoing in the Group to experimentally validate the performance of the integrated RWGSR/MeS—MCR system.

### 1.4 SUPPLEMENTAL MATERIAL

The following two reactions are considered to take place during the MeS reaction:

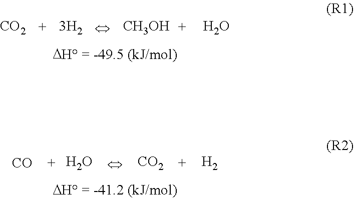

The global catalytic rate expressions for reactions R1 and R2 are presented below (Eqns 1 and 2). The thermodynamic parameters are shown in Eqns 3 to 5.

$${r}_{1} = {r}_{RWGS} = {K}_{1 w} \frac{{P}_{{CO}_{2}}}{α} [ 1 - {K}_{eqw} ( \frac{{P}_{C O} {P}_{{H}_{2} O}}{{P}_{{H}_{2}} {P}_{{CO}_{2}}} ) ] , \qquad ( 1 )$$

$$α = 1 + {K}_{2} \frac{{P}_{{H}_{2} O}}{{P}_{{H}_{2}}} + {( {K}_{3} {P}_{{H}_{2}} )}^{\frac{1}{2}} + {K}_{4} {P}_{{H}_{2} O}$$

$${r}_{2} = {r}_{MeOH} = {K}_{1 m} {P}_{{H}_{2}} \frac{{P}_{{CO}_{2}}}{{α}^{3}} [ 1 - ( \frac{1}{{K}_{eqm}} ) ( \frac{{P}_{MeOH} {P}_{{H}_{2} O}}{{P}_{{H}_{2}}^{3} {P}_{{CO}_{2}}} ) ] \qquad ( 2 )$$

$${log}_{10} {K}_{eqm} = \frac{3066}{T} - 10.5 \; 92 \qquad ( 3 )$$

$${log}_{10} 1 / {K}_{eqw} = \frac{- 2073}{T} + 2.029 \qquad ( 4 )$$

$${K}_{( i )} = A ( i ) * ( exp ( \frac{B ( i )}{R T} ) ) , i = 1 w , 1 m , 2 , 3 , 4 \qquad ( 5 )$$

Table 1.1 shows the parameter values for the global rate expressions (Eqns. (1), (2)) resulting from the fit of the experimental data [1].

TABLE 1.1
Reaction rate parameter values from the fit of the experimental data

| Kinetic | This work | | Ref. [2] | |
|---|---|---|---|---|
| constant label | A | B | A | |
| 1 m | 97.70 | 14793 | 1.07 | 36696 |
| 2 | 16581.82 | — | 3453.38 | — |
| 3 | 6.12*10⁻⁸ | 101729.2 | 0.249 | 34394 |
| 4 | 4.87*10⁻⁶ | 79412.9 | 6.62*10⁻¹¹ | 124119 |
| 1 w | 4.32*10¹⁰ | −98669.9 | 1.22*10¹⁰ | −94765 |

### 1.5 REFERENCES

[1] Z. Li, T. T. Tsotsis, Methanol synthesis in a high-pressure membrane reactor with liquid sweep, 459 J. Memb. Sci. 570-571 (2019) 103-111. https://doi.org/10.1016/j.memsci.2018.09.071.
[2] F. Zejarbad, J. Gong, Z. Li, K. Jessen, T. Tsotsis, Simulation of methanol synthesis in a membrane-contactor reactor, J. Memb. Sci. (2022) 120677 https://doi.org/10.1016/j.memsci.2022.120677.
[3] N. von der Assen, L. J. Müller, A. Steingrube, P. Voll, A. Bardow, Selecting CO₂ sources for CO₂ utilization by environmental-merit-order curves, Environ. Sci. Technol. 50 (2016) 1093-465 1101. https://doi.org/10.1021/acs.est.5b03474.
[4] A. Rafiee, K. Rajab Khalilpour, D. Milani, M. Panahi, Trends in CO₂ conversion and utilization: A review from process systems perspective, J. Environ. Chem. Eng. 6 (2018) 5771-5794. https://doi.org/10.1016/j.jece.2018.08.065.
[5] F. Samimi, N. Hamedi, M. R. Rahimpour, Green methanol production process from indirect CO₂ conversion: RWGS reactor versus RWGS membrane reactor, J. Environ. Chem. Eng. 7 (2019) 471 102813. https://doi.org/10.1016/j.jece.2018.102813.
[6] B. Doss, C. Ramos, S. Atkins, Optimization of methanol synthesis from carbon dioxide and hydrogen: Demonstration of a pilot-scale carbon-neutral synthetic fuels process, ENFUEM. 23 474 (2009) 4647-4650. https://doi.org/10.1021/ef900466u.
[7] F. Pontzen, W. Liebner, V. Gronemann, M. Rothaemel, B. Ahlers, CO₂-based methanol and DME—Efficient technologies for industrial scale production, Catal. Today. 171 (2011) 242-250. 477 https://doi.org/10.1016/j.cattod.2011.04.049.
[8] S. Dang, H. Yang, P. Gao, H. Wang, X. Li, W. Wei, Y. Sun, A review of research progress on heterogeneous catalysts for methanol synthesis from carbon dioxide hydrogenation, Catal. Today, 480 330 (2019) 61-75. https://doi.org/10.1021/acscentsci.0c00976.
[9] R. Guil-López, N. Mota, J. Llorente, E. Millán, B. Pawelec, J. L. G. Fierro, R. M. Navarro, Methanol synthesis from CO₂: A review of the latest developments in heterogeneous catalysis, 483 Materials. 12 (2019). https://doi.org/10.3390/ma12233902.
[10] C. Tisseraud, C. Comminges, T. Belin, H. Ahouari, A. Soualah, Y. Pouilloux, A. Le Valant, The Cu—ZnO synergy in methanol synthesis from CO₂, Part 2: Origin of the methanol and CO selectivities explained by experimental studies and a sphere contact quantification model in randomly packed binary mixtures on Cu—ZnO coprecipitate catalysts, J. Catal. 330 (2015) 533-488 https://doi.org/10.1016/j.jcat.2015.04.035.
[11] H. Bahruji, M. Bowker, G. Hutchings, N. Dimitratos, P. Wells, E. Gibson, W. Jones, C. Brookes, D. Morgan, G. Lalev, Pd/ZnO catalysts for direct CO₂ hydrogenation to methanol, J. 491 Catal. 343 (2016) 133-146. https://doi.org/10.1016/j.jcat.2016.03.017.
[12] A. Garciá-Trenco, A. Regoutz, E. R. White, D. J. Payne, M. S. P. Shaffer, C. K. Williams, PdIn intermetallic nanoparticles for the hydrogenation of CO₂ to methanol, Appl. Catal. B. 220 (2018) 494 9-18. https://doi.org/10.1016/j.apcatb.2017.07.069.
[13] I. U. Din, M. S. Shaharun, M. A. Alotaibi, A. I. Alharthi, A. Naeem, Recent developments on heterogeneous catalytic CO₂ reduction to methanol, J. CO₂ Util. 34 (2019) 20-33. 497 https://doi.org/10.1016/j.jcou.2019.05.036.
[14] Y. Li, W. Na, H. Wang, W. Gao, Hydrogenation of CO₂ to methanol over Au—CuO/SBA-15 499 catalysts, J. Porous Mater. 24 (2017) 591-599. https://doi.org/10.1007/s10934-016-0295-8.
[15] C. Wu, P. Zhang, Z. Zhang, L. Zhang, G. Yang, B. Han, Efficient hydrogenation of CO₂ to methanol over supported subnanometer gold catalysts at low temperature, ChemCatChem. 9 502 (2017) 3691-3696. https://doi.org/10.1002/cctc.201700872.
[16] Y. Chen, S. Choi, L. T. Thompson, Low temperature CO₂ hydrogenation to alcohols and hydrocarbons over Mo2C supported metal catalysts, J. Catal. 343 (2016) 147-156. 505 https://doi.org/10.1016/j.jcat.2016.01.016.
[17] Y. Chen, S. Choi, L. T. Thompson, Low-temperature CO₂ hydrogenation to liquid products via a heterogeneous cascade catalytic system, ACS Catal. 5 (2015) 1717-1725. 508 https://doi.org/10.1021/cs501656x.
[18] P. Konig and H. Gohna, Process of producing methanol. U.S. Patent 1995/631302A, (1995).
[19] A. Goeppert, G. A. Olah, G. K. Surya Prakash, Chapter 3.26—Toward a sustainable carbon cycle: The methanol economy, in: B. Török, T. Dransfield (Eds.), Green Chem., Elsevier, 2018: 512 pp. 919-962. https://doi.org/10.1016/B978-0-12-809270-5.00031-5.
[20] M. Specht, A. Bandi, M. Elser, F. Staiss, Comparison of CO₂ sources for the synthesis of renewable methanol, in: T. Inui, M. Anpo, K. Izui, S. Yanagida, T. Yamaguchi (Eds.), Stud Surf 515 Sci and Catal, Elsevier, 1998: pp. 363-366. https://doi.org/10.1016/S0167-2991(98)80771-4.
[21] E. R. Morgan, T. L. Acker, Practical experience with a mobile methanol synthesis device, J. 517 Sol. Energy Eng. 137 (2015) 064506. https://doi.org/10.1115/1.4031513
[22] O.-S. Joo, K.-D. Jung, 1. Moon, A. Y. Rozovskii, G. I. Lin, S.-H. Han, S.-J. Uhm, Carbon dioxide hydrogenation to form methanol via a reverse-water-gas-shift reaction (the CAMERE 520 Process), Ind. Eng. Chem. Res. 38 (1999) 1808-1812. https://doi.org/10.1021/ie9806848.
[23] S. Park, J. S. Chang, K. W. Lee, O. Joo, K. Jung and Y. Jung, Camere process for methanol synthesis from CO₂ hydrogenation, Stud Surf Sci and Catal, (2004): 67-72 523 https://doi.org/10.1016/S0167-2991(04)80221-0.
[24] T. Matsushita, T. Haganuma and D. Fujita, US. Patent 2013/0237618A1, (2013)
[25] B. Stefansson, Methanol fuel from power and CO₂ emissions, 2015 European Methanol Policy Forum Brussels, Oct. 14, (2015).
[26]0. F. Sigurbjörnsson, Recycling of geothermal carbon and sulfur emissions for chemical production, (2013).
[27] F. S. Zebarjad, S. Hu, Z. Li, T. T. Tsotsis, Experimental investigation of the application of ionic liquids to methanol synthesis in membrane reactors, Ind. Eng. Chem. Res. 58 (2019) 11811-531 11820. https://doi.org/10.1021/acs.iecr.9b01178.
[28] V. Dieterich, A. Buttler, A. Hanel, H. Spliethoff, S. Fendt, Power-to-liquid via synthesis of methanol, DME or Fischer-Tropsch-fuels: a review, Energy Environ. Sci. 13 (2020) 3207-3252. 534 https://doi.org/10.1039/dOee01187h.
[29] T. T, Tsotsis and K. Jessen, A novel reactive separation method for carbon dioxide capture from flue gas, U.S. Department of Energy National Energy Technology Laboratory, Research Performance Progress Report, Aug. 30, 2021.
[30] L. Pastor-Pérez, F. Baibars, E. Le Sache, H. Arellano-Garciá, S. Gu, T. R. Reina, CO₂ valorisation via reverse water-gas shift reaction using advanced Cs doped Fe—Cu/Al₂O₃ catalysts, 540J. CO₂ Util. 21 (2017) 423-428. https://doi.org/10.1016/j.jcou.2017.08.009.
[31] L. Pastor-Pérez, M. Shah, E. Le Saché, T. Ramirez Reina, Improving Fe/Al₂O₃ catalysts for the reverse water-gas shift reaction: On the effect of Cs as activity/selectivity promoter, Catalysts. 543 8 (2018) 608. https://doi.org/10.3390/catal8120608.
[32] J. M. Moe, Design of water-gas shift reactors, Chem. Eng. Prog. 58 (1962) 33-545 36. https://doi.org/10.1252/jcej.08we186.
[33] F. Sadat Zebarjad, Z. Wang, H. Li, S. Hu, Y. Tang, T. T. Tsotsis, Investigation of CO₂ and Methanol Solubility at High Pressure and Temperature in the Ionic Liquid [EMIM][BF4] Employed During Methanol Synthesis in a Membrane-Contactor Reactor.” Chem. Eng. Sci., 241, 549 116722, 2021.

### 2. COMPARATIVE ANALYSIS

Four technologies are examined in this section: (1) the CAMERE process that involves the conversion of CO₂ via the reverse-water gas shift reaction into a syngas mixture followed by the conversion of such syngas into MeOH via a conventional MeS reactor; (2) the direct catalytic conversion of CO₂ into MeOH, which is the process practiced commercially by CRI; (3) the indirect electrochemical method, which combines the electrochemical conversion of CO₂ with a conventional catalytic MeS reactor; and (4) the direct electrochemical conversion of CO₂ into MeOH. We compare the five CCU processes on the assumption of a 95% carbon conversion efficiency (i.e., carbon capture rate), and evaluate the feasibility of each technology on the basis of its minimum selling price (MSP), net present value (NPV), and discounted payback period (DPBP), as well as energy efficiency. We conduct, in addition, a sensitivity analysis examining the effect of renewable H₂ and CO₂ costs and plant capacity.

### 2.1. Methods

### 2.1.1 Process Design

The process flow diagrams for each technology are shown in FIG. 11. FIG. 11a represents the RWGSR/MeS-MCR, FIG. 11B the CAMERE. FIG. 11C the direct CO₂-to-MeOH, FIG. 11D the indirect electrochemical, and FIG. 11E the direct electrochemical technologies. The various relevant technical parameters and operating conditions utilized in the study have been extracted from commercial and technical literature pertaining to each of these technologies. Material and energy balances were generated using the AVEVA Process Simulation software, and the TEA was conducted using the AVEVA Process Simulation economic package. More detailed information related to the assumptions made and the design characteristics of each of the technologies is provided in sections 2.1.1 to 2.1.5 below.

### 2.1.1.1 the RWGSR/MeS-MCR Technology

This is a novel direct CO₂ into the MeOH conversion process [27, 28, 30] that overcomes the limitations faced by other current CO₂-based MeS technologies. In this process, H₂ produced by a PEM electrolyzer (cell voltage=2.4 V and current density=1.5 A/cm²) [32] is mixed with CO₂ (H₂/CO₂ molar ratio=3), and the resulting mixture is heated and fed into an RWGSR which converts CO₂ into CO (operating conditions for the RWGSR, T=600° C., P=1 bar, and conv 60%). The water electrolysis reaction in the PEM electrolyzer (Equation R1) [33] and the RWGS reaction (Equation R2) [34] are shown below:

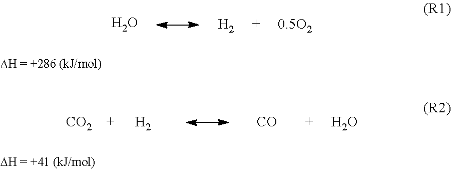

In the next step, the water produced by the RWGS reaction is removed from the gas steam exiting the RWGSR using a condenser, and the dried syngas is then compressed and fed to the shell side of a MeS-MCR (operating conditions T=220° C., P=30 bar) where it is converted into MeOH via reactions R3 and R4 below.

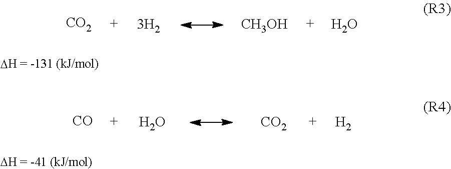

Simultaneously, a solvent is pumped through the tube-side (membranes) of the reactor, acting both as a coolant and as a sweep stream to remove in situ the reaction products. Removing the MeOH and water produced by the catalytic reaction from the reactor shell-side and transporting them through the membrane into the reactor tube-side, where they are dissolved in the sweep solvent, helps to overcome the thermodynamic limitations associated with the MeS reaction, and to attain conversions of ~80% or higher. In the next step, the unreacted syngas is separated from the MeOH/water mixture in a condenser and recycled back to the RWGSR, and the sweep solvent is regenerated. Finally, the MeOH/water mixture is fed into a distillation column to produce 99.8% pure MeOH as the final product.

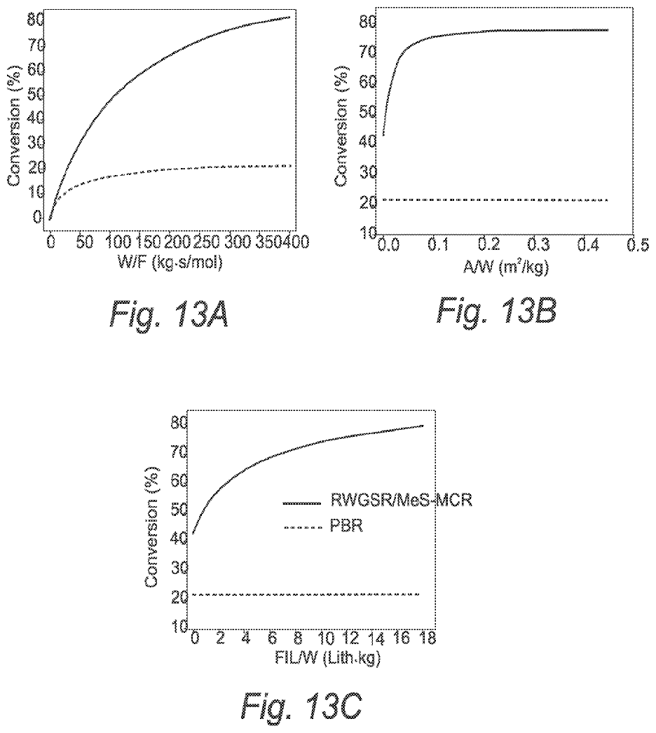

FIG. 13 shows simulation results of the behavior of a RWGSR/MeS-MCR system employing an experimentally validated model, and for comparison purposes, the Figure also shows the behavior of a conventional PBR processing pure CO₂ at a temperature of 250° C. and a pressure 80 bar (which are the conditions employed by the CRI process, see Sect. 2.1.1.3). The feed to both reactors consists of a H₂/CO₂ mixture (molar ratio=3), The RWGSR operates at a pressure of 1 bar and a temperature of 600° C., attaining equilibrium conversion. The MeS-MCR operates at a temperature of 220° C., and a pressure of 30 bar. FIG. 13A shows the effect of the ratio of the mass of MeS catalyst to the total molar feed flow rate, W/F, on carbon conversion in both the RWGSR/MeS-MCR and the PBR systems. By increasing the W/F to ~200, the PBR reaches equilibrium, and further increasing the W/F does not improve its conversion. On the other hand, the conversion in the RWGSR/MeS-MCR continues to increase with increasing W/F since the product removal in the MCR helps to overcome the equilibrium limitations problem. For a sufficiently large W/F, the RWGSR/MeS-MCR conversion is almost four times that of the PBR. This improvement in conversion is important, since it significantly reduces the size of the required recycle stream (needed to attain 95% carbon conversion), thus allowing the use of smaller and more efficient unit operations in MeOH plants.

FIG. 13B shows the carbon conversion in the RWGSR/MeS-MCR as a function of membrane surface area per MeS catalyst mass, A/W, (in these simulations, we keep the mass of the catalyst constant and vary the membrane area), along with PBR conversion employing the same quantity of MeS catalyst, for comparison purposes. The RWGSR/MeS-MCR conversion increases sharply with increasing the A/W, since a higher membrane surface provides for increased mass transfer, with conversion reaching 80% asymptotically for large membrane areas, while the PBR conversion stays constant at −22%. Finally, FIG. 13C shows the RWGSR/MeS-MCR conversion as a function of sweep liquid flow rate per catalyst mass (Fa/W) (in these simulations, we keep the mass of the MeS catalyst constant and vary the flow rate of the solvent) along with the PBR conversion employing the same quantity of catalyst, for comparison purposes. Increasing the sweep solvent flow rate significantly increases the RWGSR/MeS-MCR conversion by helping maintain high the driving force for MeOH mass transfer through the membrane. The sweep liquid is being circulated in the system using a pump; therefore, higher Fn/W can be achieved by just increasing the pumping speed, and the purchase of extra liquid is not required. Thus, a FtJW in the range of 14-18 is a good choice, resulting in a RWGSR/MeS-MCR conversion approximately four times higher than that of the PBR.

### 2.1.1.2 the CAMERE Technology

A two-stage process for CO₂ conversion into MeOH (named CAMERE), combining a RWGSR with a conventional MeS reactor, was developed by the Korean Institute of Science and Technology [22, 23]. In this process, H₂ produced by a PEM electrolyzer (cell voltage=2.4 V, and current density=1.5 A/cm²) is mixed with CO₂ (molar ratio H₂/CO₂=3), with the mixture heated and then fed to the RWGSR which converts the CO₂ into CO (operating conditions T=600° C., P=1 bar, and conv 60%). In the next step, the produced water in the RWGSR is removed from the reactor's exit stream via a condenser, and the dried syngas is then compressed and fed to a conventional MeS reactor (T=250° C., P=80 bar, and conv 50%). In the next step, the unreacted syngas is separated from the MeOH/water mixture in a condenser and recycled back to the RWGSR. Finally, the MeOH/water mixture is fed to a distillation column to produce a 99.8% pure MeOH stream as the final product.

### 2.1.1.3 the CRI Direct CO₂-to-MeOH Synthesis Technology

In 2014, CRT reported a commercial-scale CO₂-to-MeOH plant in Svartsengi, Iceland [35]. The plant converts CO₂ into MeOH in a single step in a conventional catalytic MeS reactor. In this process, H₂ produced by a PEM electrolyzer (cell voltage=2.4 V, and current density=1.5 A/cm²) is mixed with CO₂ (molar ratio H₂/CO₂=3), heated, and then fed into the conventional MeS reactor, which converts CO₂ into MeOH (operating conditions T=250° C., P=80 bar, and conv 22%). In the next step, the unreacted syngas is separated from MeOH/water in a condenser and recycled back to the MeS reactor. Finally, the MeOH/water mixture is fed to a distillation column to produce a 99.8% pure MeOH product.

### 2.1.1.4 the Indirect Electrochemical Conversion of CO₂ to MeOH

This technology consists of two key process steps, which include H₂ and CO production in two separate electrolyzers, followed by a conventional catalytic MeS reactor. Specifically, a PEM electrolyzer (cell voltage=2.4 V, and current density=1.5 A/cm²) is used for H₂ production from water and, in parallel, an alkaline electrolyzer (cell voltage=7.4 V, current density=0.3 A/cm², and Faradaic efficiency 100%) is used to convert CO₂ into CO [36, 37], with the following reaction taking place in the CO₂-to-CO electrolyzer [38]:

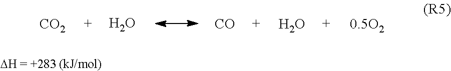

The produced syngas containing CO, CO₂, and H₂ is then heated, compressed, and fed to the conventional MeS reactor, which converts the syngas to MeOH (operating conditions T=250° C., P=80 bar). In the next step, the unreacted syngas is separated from MeOH/water in a condenser and recycled back into the MeS reactor. Finally, the produced MeOH/water mixture is fed to a distillation column to produce 99.8% pure MeOH as the final product.

2.1.1.5 the Direct Electrochemical Conversion of CO₂ into MeOH

This technology directly converts the CO₂ into MeOH in a one-step electrolyzer system. Specifically, water and CO₂ are fed into an alkaline electrolyzer (cell voltage=4.4 V, current density=0.1 A/cm², and Faradaic efficiency 80%) in which CO₂ is directly converted into MeOH according to the following reaction [39]:

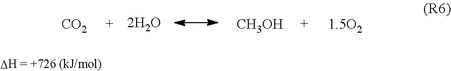

In the next step, the unreacted syngas is separated from the MeOH/water mixture in a condenser and is recycled back to the electrolyzer. The MeOH/water mixture from the condenser is fed into a distillation column to produce 99.8% pure MeOH as the final product.

### 2.1.2 Operating Costs

Renewable electricity is utilized in all five cases. The electricity cost is assumed to be $68/MWh, consistent with the average price of solar energy in 2019 [40]. The baseline cost of captured CO₂ is assumed to be $30/ton [26]. In this TEA study, we assume using a pure CO₂ feed stream and we do not specify the source of that CO₂. Note, however, that the RWGSR/MeS-MCR process, in contrast to the other technologies, in addition to pure CO₂, can also directly utilize other flue gas streams without needing an intervening CO₂ separation step, and this represents a key advantage over the competing technologies. Nevertheless, in the TEA study presented here we do not make such a claim and we compare, instead, the five technologies on the basis of the same CO₂ cost. The baseline cost for renewable H₂ is taken to be equal to $4.5/kg [41]. The baseline CO₂ feed flow rate (determining the plant size) is taken equal to 86.8 kmol/h, while the H₂/CO₂ feed molar ratio is set equal to 3.

For the RWGSR/MeS-MCR process, the baseline membrane cost is assumed to be $400/m², with a five-year membrane lifetime as the baseline case. We assume a baseline cost for the sweep solvent used by the process (in our experimental studies, we employed two different types of solvents, an ionic liquid (IL) and TGDE, a petroleum-derived liquid) equal to $200/kg. We assume the costs for the RWGSR and MeS-MCR catalysts to be the same and equal to $12.9/kg [42].

In addition to the baseline cost value for the various parameters (CO₂, H₂ and solvent costs, membrane cost and lifetime, and plant size), we have also studied a range of other values of these parameters, as further discussed in the paper below, in order gauge their influence of process performance.

TABLE 1
Baseline parameter values.

| Parameter | Baseline value | Unit |
|---|---|---|
| Cost of renewable | 68 | $/MWh |
| electricity | | |
| Cost of renewable H₂ | 4.5 | $/kg |
| CO₂ cost | 30 | $/ton |
| Membrane cost | 400 | $/m² |
| Membrane lifetime | 5 | year |
| Solvent cost | 200 | $/kg |
| CO₂ feed flow rate | 86.8 | kmol/h |

For all five processes studied here, the carbon efficiency (i.e., CO₂ capture rate) is set equal to 95%. This can be accomplished technically by controlling the flow rate of the vent stream. In calculating the process economics, we assume a 30-year plant life, a 21% annual tax rate, an 8% annual interest rate, and a 2% annual inflation rate [10], see Table 2 for a summary. The experimental operating conditions (i.e., temperature and pressure) for the catalytic reactors for the four processes that employ such reactors are summarized in Table 3, while the parameters used and the assumptions made for the calculations relating to the electrolyzers are summarized in Table 4.

TABLE 2
Summary of financial parameters.

| Parameter | Value |
|---|---|
| Plant construction time | 3 years |
| Plant life | 30 years |
| Annual tax rate | 21% |
| Annual interest rate | 8% |
| Annual inflation rate | 2% |

TABLE 3
Summary of operating conditions for the catalytic reactors.

| | Reactor | Reactor |
|---|---|---|
| Process | temperature (° C.) | pressure (bar) |
| RWGSR/MeS-MCR | 600 for the RWGSR | 1 for the RWGSR |
| | 220 for MeS-MCR | 30 for the MeS-MCR |
| CAMERE | 600 for the RWGSR | 1 for the RWGSR |
| | 250 for the MeS reactor | 50 for the MeS reactor |
| Direct CO₂-to-MeOH | 250 | 80 |
| Indirect | 250 for the MeS reactor | 80 for the MeS reactor |
| electrochemical | | |

The goal of this TEA study is to determine the plant's profitability by calculating key financial performance indicators such as the NPV, the DPBP, and the MSP, which are computed here by the following equations:

$$NPV = {\sum }_{j = 1}^{planlife} ( \frac{{CF}_{j}}{{( 1 + r )}^{j}} ) - TCI \qquad ( 1 )$$

$$TCI = {\sum }_{j = 1}^{DPBP} ( \frac{{CF}_{j}}{{( 1 + r )}^{j}} ) \qquad ( 2 )$$

Here CF, r, and TCI are the cash flow, discount rate, and total capital investment, respectively. The MSP is calculated as the MeOH price for which the corresponding NPV is equal to zero [46].

TABLE 4
The parameters used in the calculations
of the various electrolyzers.

| Variable | value | ref |
|---|---|---|
| PEM H₂O electrolyzer | | |
| Stack capital cost ($/m²) | 10,000 | [43] |
| Current density (A/cm²) | 1.5 | [32] |
| Cell voltage (V) | 2.4 | [32] |
| Faradaic efficiency (%) | 100 | [26] |
| Alkaline CO₂-to-CO electrolyzer | | |
| Stack capital cost ($/m²) | 1840 | [43] |
| Current density (A/cm²) | 0.3 | [36] |
| Cell voltage (V) | 7.4 | [37] |
| Faradaic efficiency (%) | 100 | [36] |
| Alkaline CO₂-to-MeOH electrolyzer | | |
| Stack capital cost ($/m²) | 1840 | [43] |
| Current density (A/cm²) | 0.1 | [44] |
| Cell voltage (V) | 4.4 | [45] |
| Faradaic efficiency (%) | 80 | [44] |

### 2.2. RESULTS AND DISCUSSION

### 2.2.1 MSP Calculations for the Baseline Cases

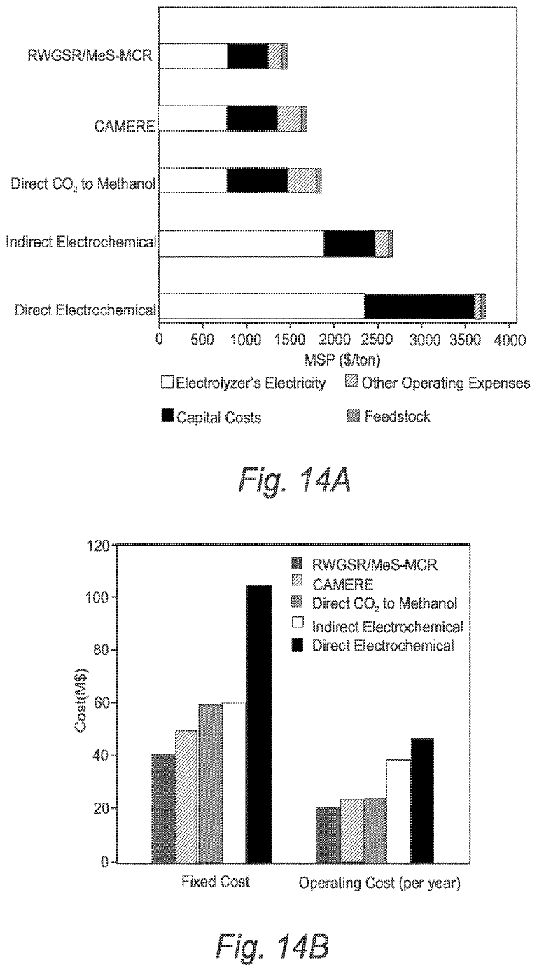

For each of the five CO₂-to-MeOH technologies studied here, we have calculated the MSP (according to the method discussed in section 2.1.2) corresponding to the baseline parameters shown in Table 1. For all cases, we have employed the same economic parameters and assumptions shown in Table 2. The MSP for the RWGSR/MeS-MCR, CAMERE, direct CO₂ to MeOH, indirect electrochemical, and direct electrochemical technologies are (see FIG. 14A) 1443, 1687, 1872, 2605, and 3682 $/ton, respectively, showing that the proposed RWGSR/MeS-MCR technology is more economical than the other four technologies. Notably, all three thermochemical technologies are significantly more economical than the two electrochemical processes. FIG. 14A also shows the contribution that the various cost factors, which include the electricity used for the operation of the electrolyzers and the capital, feedstock, and operating costs, make towards determining the MSP for each of the five processes. For all five technologies and, in particular, for the two electrochemical technologies, the cost of the electricity for the operation of the electrolyzer(s) is the dominant contributor to the MSP. This means that designing more energy-efficient electrolyzers or producing renewable electricity at a lower cost has the potential to significantly decrease the MSP of all technologies and should, thus, motivate further R&D for the development of energy-efficient electrolyzers and for cost-efficient renewable electricity production. It should be noted that the cost for the electrolyzer operation is the same for the three thermochemical technologies since they are all assumed to use a PEM electrolyzer using the same quantity of feed water. The Indirect Electrochemical process uses an alkaline electrolyzer for converting the CO₂ into CO as well as a PEM electrolyzer for H₂ production, and its electrolyzer-related operating costs are higher than those of the thermochemical processes. The electrolyzer operating cost for the Direct Electrochemical process is the highest among the five technologies. This is because producing 1 mole of MeOH requires 6 moles of electrons. Moreover, the specific electrolyzer utilized employs a relatively high cell voltage of 4.4 V which results in high electricity consumption.

In terms of the impact of capital costs on the MSP, the RWGSR/MeS-MCR process has the lowest associated costs. This is because, through the use of the MeS-MCR, the process attains conversions of ~80% (as assumed in this study) or higher which, in turn, significantly diminishes the magnitude of the recycle flow rate needed to attain the desired carbon conversion rate into MeOH (as can be seen in Table 5, which lists the required recycle molar flow rates for all five processes).

TABLE 5
Calculated recycle flow rates for the five
technologies under the baseline conditions.

| Process | Recycle flow rate (kmol/h) |
|---|---|
| RWGSR/MeS-MCR | 84.8 |
| CAMERE | 691.8 |
| Direct CO₂ to MeOH | 1270.2 |
| Indirect Electrochemical | 831.2 |
| Direct Electrochemical | 84.4 |

The MeS-MCR technology benefits, as a result, from having smaller and more economical sub-units. The capital cost contribution to determining the MSP is the highest among all technologies for the Direct Electrochemical process even though the technology also requires a low recycle flow rate (see Table 5). This is because it utilizes a special type of alkaline electrolyzer with a low current density (0.1 A/cm²). Thus, a large stack surface area is required for converting the desired amount of CO₂ into MeOH which, in turn, implies a high capital cost for the electrolyzer.

The operating expenses, other than the cost of electricity for the electrolyzer, are higher (see FIG. 14A) for the thermocatalytic processes compared to the electrochemical ones, as expected, since they have greater energy requirements for heating the catalytic reactors and for compressing the gas in the process. Among the three thermocatalytic technologies, the RWGSR/MeS-MCR process has the lowest operating cost, and the Direct CO₂ to MeOH technology has the highest one, which directly correlates with the conversion of the MeOH synthesis reactors employed in these processes and the associated required recycle flow rates. As expected, the lower the conversion and the higher the recycle flow rate, the higher the corresponding operating costs. The costs for the feedstocks (CO₂ and H₂O) are the same for all three processes, as expected, and represent only a small fraction of the total MSP.

FIG. 14B presents the fixed cost and annual operating costs for each of the five CO₂ to MeOH technologies. The RWGSR/MeS-MCR technology has the lowest estimated fixed ($40.7M) and annual operating cost ($20.8M). This is because the use of the MeS-MCR helps the technology to be more cost and energy efficient, with a small recycle flow rate needed (in general, the higher the recycle flow rate, the higher the operating cost required for processing this stream). On the other hand, the Direct Electrochemical technology has the highest fixed cost ($104.6M) and annual operating cost ($46.9M) since it employs an expensive alkaline electrolyzer with large electricity consumption, as previously noted.

### 2.2.2 Energy Calculations for the Baseline Cases

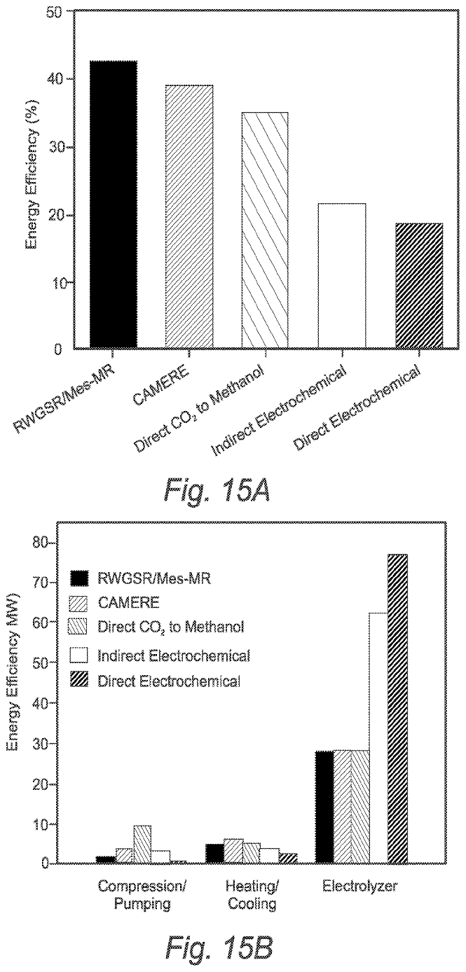

The energy efficiency for each of the five processes (under the baseline conditions of Table 1) is determined by dividing the total energy contained in the MeOH product (based on its lower heating value or LHV) by the total energy input into the system. The key energy inputs are categorized as a) the energy required for compression and pumping, b) the energy required for heating and cooling, and c) the energy consumed in the electrolyzers. FIG. 15A shows that the RWGSR/MeS-MR process, with an estimated energy efficiency of 42.7%, is more energy efficient than the CAMERE (38.8%), the Direct CO₂ to MeOH (34.8%), the Indirect Electrochemical (21.4%), and the Direct Electrochemical (18.7%) processes. FIG. 15B further explains the differences in energy efficiency among the five processes by showing the energy consumption breakdown for each technology.

The energy consumed by the electrolyzers is dominant for all five technologies, demonstrating the importance of designing more energy-efficient electrolyzers. The three thermocatalytic technologies (RWGSR/MeS-MCR, CAMERE, and Direct CO₂ to MeOH) use the same PEM electrolyzer for H₂ production and have, as a result, the same electrolyzer-related energy consumption (34.5 MW). The Indirect Electrochemical technology consumes a significantly higher amount of electrolyzer-related electricity (58.4 MW) compared to the thermocatalytic technologies since, in addition to using a PEM electrolyzer for H₂ production, it also uses an alkaline CO₂-to-CO electrolyzer with a high cell voltage of 7.4 V and a relatively low current density of 0.3 A/cm² (see Table 4) that consumes a considerable amount of electricity to convert CO₂ into CO, thus resulting in high energy consumption and correspondingly low energy efficiency.

The Direct Electrochemical process has the highest electrolyzer-related electricity consumption and the lowest energy efficiency among all technologies because it employs an alkaline CO₂-to-MeOH electrolyzer with a relatively high cell voltage of 4.4 V and a poor current density of 0.1 A/cm². The CAMERE technology has the highest energy consumption related to heating/cooling (6.2 MW), because the process utilizes an RWGSR operating at 600° C. that requires a high heating energy input. The RWGSR/MeS-MCR technology also uses an RWGSR operating at 600° C., however, since the high conversion in the MCR results in a lower required recycle flow rate, the combined feed flow rate to the RWGSR is lower compared to the one for the CAMERE, thus resulting in lower energy consumption needed for providing heat to the reactor. For the Direct CO₂ to MeOH technology, because of the low conversion in the MeS reactor resulting in a large recycle flow rate, the energy required for the operation of the condensers represents a major component of the heating/cooling-related energy consumption. For the two electrochemical technologies, the heating/cooling-related energy consumption mostly relates to the distillation columns. The Direct CO₂ to MeOH technology has a considerably higher energy consumption in the compression/pumping section (9.4 MW) because the MeS reactor utilized in this technology operates a high pressure of 80 bar and has a low conversion of −25% as well, thus requiring a very large recycle flow rate.

### 2.2.3 Effect of CO₂ Cost on MSP

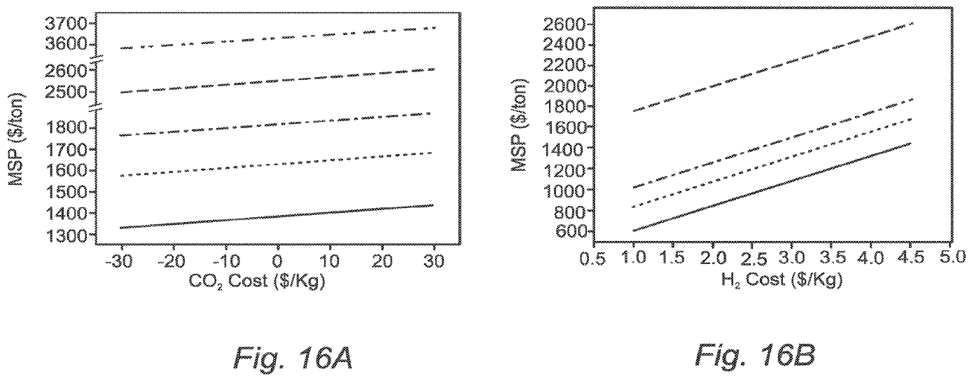

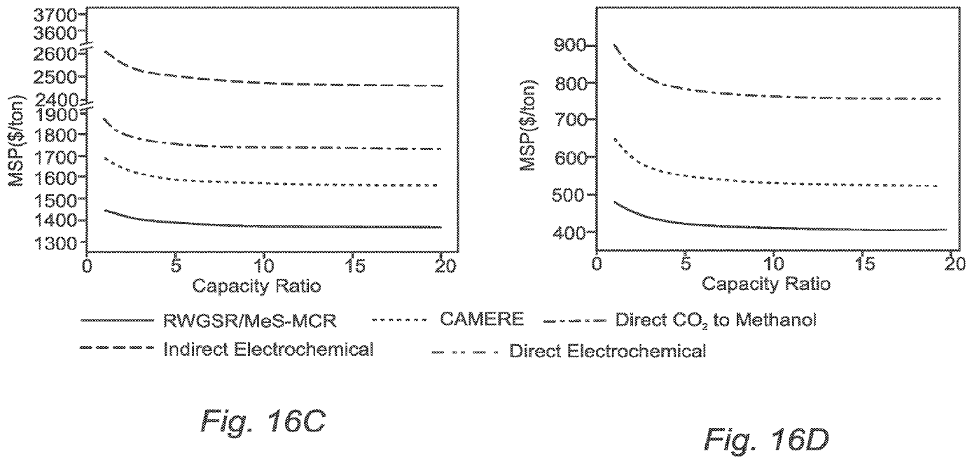

The results of the analysis of the impact of CO₂ cost (with all other parameters being kept at their baseline values) on the MSP of the five different processes are shown in FIG. 16A. We have extended the analysis down to negative CO₂ costs on the assumption that future carbon capture and management programs implemented by various governments may offer financial incentives for the conversion of this environmentally harmful component into a green fuel like MeOH. As shown in FIG. 16A, for the whole range of CO₂ costs considered, the RWGSR/MeS-MR technology has a lower MSP compared to the other four technologies studied.

### 2.2.4 Effect of the Cost of Renewable H₂ on MSP

The three thermocatalytic and the Indirect Electrochemical processes make use of renewable H₂. Here, we analyze the impact on MSP of the cost of such H₂ (with all other factors affecting the MSP being kept constant at their baseline values). We investigate a range of costs, from $4.5/Kg (the baseline value) down to $1/Kg, with the lower end matching the optimistic target set by the U.S. Department of Energy (DOE) for 2030 [47]. The results of such analysis are presented in FIG. 16B. For the whole range of renewable H₂ costs studied, the RWGSR/MeS-MCR technology has a lower MSP compared to the other two technologies. For a renewable H₂ cost of −$0.7/Kg, the MSP for the RWGSR/MeS-MCR technology matches the current selling price for MeOH of $500/Kg [48]. These findings clearly show the critical importance of lowering the renewable H₂ production costs on the economics of all the CO₂ conversion processes studied here. Clearly, the cost of H₂ has a more substantial impact on the process MSP, as expected, based on the molecularity of the MeS reaction that requires three moles of H₂ per mole of CO₂ converted.

### 2.2.5 Effect of Plant Capacity on the MSP

Another key factor determining the MSP is the plant size. FIG. 16C shows the results of such analysis for the five CO₂ conversion processes where the MSP is plotted as a function of the ratio of the plant size with respect to the baseline plant size (86.8 kmol/h of CO₂ feed flow rate), with all other factors affecting the MSP (other than the plant size) being kept constant at their baseline values. The MSP of the RWGSR/MeS-MR technology remains lower than that of the other four processes for the whole range of plant capacities investigated. For all technologies, there is approximately a 5 to 10% drop in MSP from the baseline plant size to the largest (20×) plant size investigated. This relatively small improvement in MSP with plant size scale-up is consistent with the discussion in Section 2.2.1 (see FIG. 14), showing that the operating costs have more of an impact on MSP than the fixed costs associated with the plant capacity.

The magnitude of the impact of plant scale-up on MSP depends, however, on the other process parameters. For example, in FIG. 16D we plot the MSP for the three thermocatalytic processes as a function of the ratio of the plant size with respect to the baseline plant size for the case for which the CO₂ cost is assumed to be $−30/ton (e.g., due to government carbon tax incentives), and the cost of renewable H₂ is $1/kg (meeting DOE's ambitious target for 2030) with all other parameters remaining at their baseline values. As shown in FIG. 16D, the drop in MSP from the baseline plant size to the largest (20×) plant size investigated ranges from 18.3% for the RWGSR/MeS-MCR technology to 24.1% for the CAMERE process and 19.3% for the Direct CO₂ conversion process. This is because the dominance of operating over fixed costs is significantly less so for FIG. 16D than for FIG. 16C. Note further that the MSP for the RWGSR/MeS-MCR process falls in the range of commercial MeOH market costs ($300 to $600/kg) [49], highlighting the fact that the technology may become, in the near future, a close competitor to the existing conventional MeS technology while being more energy efficient and environmentally friendly.

### 2.2.6 DPBP and NPV Analysis

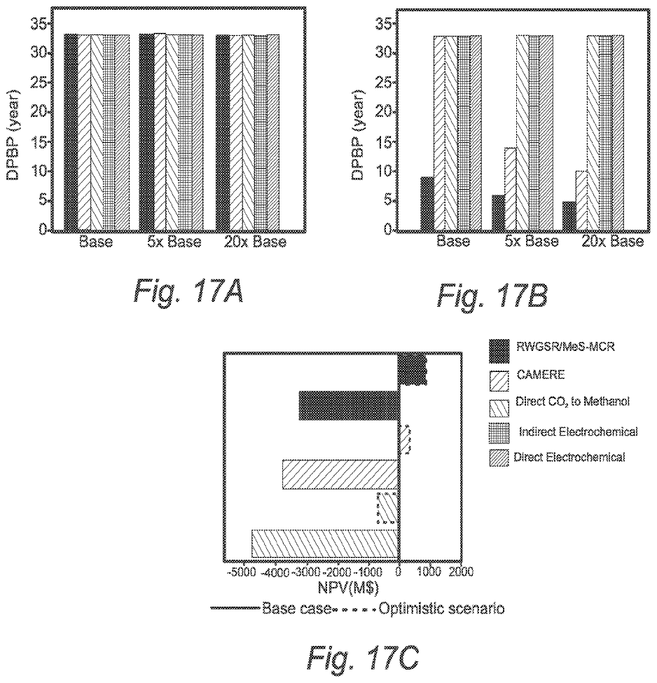

FIG. 17A shows the calculated DPBP for the RWGRS/MeS-MCR and the other four CO₂ to MeOH technologies. As with FIG. 16D, we assume the CO₂ cost to be $−30/ton (due to government carbon tax incentives), and the cost of renewable H₂ to be $1/kg with all other parameters remaining at their baseline values. For the analysis in FIG. 17A, we assume the MeOH selling price to be $400/ton, and we study three different plant sizes: the baseline case and two other plants, one with a capacity 5× larger and another with a capacity 20× larger than the baseline plant. As shown in FIG. 17A, none of the five technologies, when the MeOH selling price is set at $400/ton, will be profitable in the 33 years of the project lifespan (3 years of construction plus 30 years plant lifetime), and their cumulative discounted cash flow is always negative, even under the somewhat optimistic scenario of a CO₂ cost of $−30/ton and a renewable H₂ cost of $1/Kg.

The picture becomes a bit “brighter” when the MeOH selling price is set at $600/ton, see FIG. 17B. For the baseline size plant, the RWGSR/MeS-MCR technology with the DPBP of 9 years is still the only technology among the five technologies that is feasible in this plant size range. When higher plant capacities, i.e., 5× and 20× of baseline, are considered, the CAMERE technology also becomes feasible. However, the RWGSR/MeS-MCR technology has the lowest DPBP for all the different plant capacities studied, indicating that this technology would be the most profitable. The Direct CO₂ to MeOH thermocatalytic and the two electrochemical technologies will not become profitable over the 33 years of the project lifespan, and their cumulative discounted cash flow is always negative, showing that these technologies will not be competitive under the scenario of a CO₂ cost of $−30/ton, and renewable H₂ cost of $1/kg, even when the MeOH selling price is set at $600/kg.

A comparative study for the NPV among the three thermocatalytic processes, considering a plant with a size 20× larger than the baseline capacity and a MeOH selling price set at 600 $/kg, is presented in FIG. 17C. The analysis is conducted under both the baseline conditions (Table 1) and also under the scenario of a CO₂ cost of $−30/ton, and renewable H₂ cost of $1/kg. FIG. 17C shows that none of the three processes can be profitable for the baseline case since their respective NPVs are negative. However, under the scenario of a CO₂ cost of $−30/ton and a renewable H₂ cost of $1/kg, the RWGSR/MeS-MCR and CAMERE processes, with calculated NPVs of $831M and $320M, respectively, become profitable and financially feasible. An interesting thing to note is that under the latter conditions, the MSP for the CAMERE technology ($525.4/ton) is 29.4% higher than that of RWGSR/MeS-MCR technology ($406.1/ton); however, the calculated NPV for RWGSR/MeS-MCR technology ($831M) is 2.6 times higher than that of CAMERE technology ($320M). This indicates that even a moderate difference between the MSP's among two different technologies can lead to significant differences in profitability, as attested by their NPV factors.

### 2.2.7 Effect of Membrane and Sweep Solvent Cost on MSP

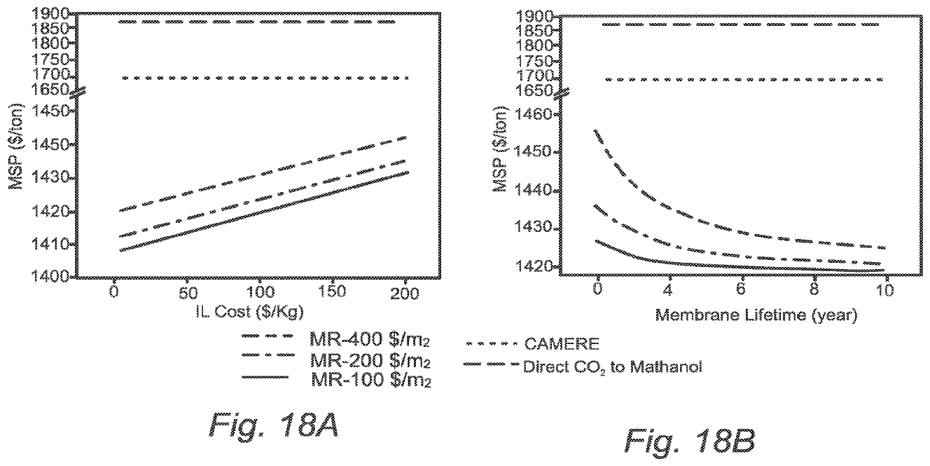

A sensitivity analysis has been conducted on the effect on the MSP of the RWGSR/MeS-MR technology of the cost of the sweep solvent and membrane and of membrane lifetime, with the rest of the parameters kept at their baseline values. The analysis results are presented in FIG. 18, where the MSP for the other two thermocatalytic (CAMERE and Direct CO₂) processes (which do not depend on these costs) are also shown for comparison purposes. The rate of sweep solvent loss in these simulations is assumed to be 0.1 g solvent per kg of produced MeOH, consistent with our experimental findings. We have studied a range of solvent costs from $5/Kg to $200/Kg, and a range of membrane costs from $100/m² to $400/m² to account for the price range of a wide variety of sweep solvents and membranes that can be used in this technology. As an example, we have previously studied the performance of petroleum-based solvents (TGDE), as potential alternatives for ionic liquids (IL), in MeS-MCR for MeOH. These petroleum solvents are much cheaper than the IL, and their price is close to the lower bound considered ($5/kg). The upper end of the range ($200/Kg) is a realistic estimate for the bulk-volume price of the IL's. For example, the wholesale price (for purchases of more than 1 ton) for the ionic liquid [EMIM][BF₄] that is currently being used in our studies is ~$70/Kg. The same is true for the membrane cost, with the cost range of $100-$400/m² intended to cover different types of membranes currently available.

FIG. 18A shows the effect of sweep solvent price and membrane cost on the MSP of the RWGSR/MeS-MR technology (assuming a membrane lifetime of 5 years). Shown in the Figure are also the MSP for the CAMERE and Direct CO₂ to MeOH technologies. The MSP for the baseline case (solvent cost=$200/kg and membrane cost=$400/m²) is $1443/ton and decreases with decreasing solvent and membrane cost. For the case of the solvent cost=$5/kg and the membrane cost=$100/m², the MSP is $1408/ton, which is 19.8%, and 32.9% lower than the MSP for the CAMERE and the Direct CO₂ to MeOH technologies, respectively. FIG. 18B shows the effect of membrane lifetime on the MSP of the RWGSR/MeS-MR technology. A longer lifetime means that the membranes need to be replaced less frequently, lowering the cost of purchasing new membranes and, thus the MSP.

### 2.3. CONCLUSIONS

The motivation to develop efficient CO₂-to-MeOH technologies arises from today's urgent need to effectively manage the emissions of this key greenhouse gas. Our research group recently introduced a novel CCU technology for the production of MeOH from waste CO₂ [31]. The process employs a novel configuration that combines a reverse water gas shift reactor (as stage 1) with a membrane contactor MeS reactor (stage 2) that our team previously developed for the conversion of dilute syngas mixtures into MeOH. This integrated RWGSR/MeS-MCR process overcomes the challenges that direct CO₂ conversion into MeOH faces. In the RWGSR stage, the waste CO₂ is converted into a syngas mixture for use in the MeS-MCR stage. Employing the MeS-MCR system addresses the equilibrium limitations that the MeS reaction faces by removing reaction products in situ using a sweep solvent and achieving carbon conversion rates exceeding 80%, a performance that significantly surpasses the equilibrium conversion.

In this study, a comparative TEA was conducted of the RWGSR/MeS-MCR process along with four other CO₂-to-MeOH CCU processes. Key economic parameters, including the MSP, NPV, DPBP, as well as energy efficiency, were analyzed with respect to CO₂ and renewable H₂ costs and plant capacity, and for the RWGSR/MeS-MCR process with respect to membrane and sweep liquid costs. The study revealed that the RWGSR/MeS-MCR technology has the highest energy efficiency and the lowest MSP among all the five technologies investigated. This is due to the fact that integrating the RWGSR and MeS-MCR subsystems together allows the process to attain carbon conversions of 80% or higher, resulting in a significantly reduced recycle flow rate, which means that smaller and more energy-efficient sub-units are needed. Although all five technologies offer the significant societal benefit of being able to directly capture and utilize waste CO₂, a key greenhouse gas, in terms of their MSP under the baseline conditions for CO₂ and renewable H₂ costs, none of them can currently compete with conventional MeS. However, under an optimistic future scenario whereby government incentives lower the CO₂ cost and technological advances lower the price of renewable H₂, both the proposed RWGSR/MeS-MCR and another catalytic technology (CAMERE) become price competitive.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### 2.4 REFERENCES

[1] Sayed, E. T., et al., Renewable energy and energy storage systems. Energies, 2023. 16(3): p. 1415.
[2] Makiela, K., B. Mazur, and J. Glowacki, The impact of renewable energy supply on economic growth and productivity. Energies, 2022. 15(13): p. 4808.
[3] Tabarkhoon, F., et al., Synthesis of novel and tunable Micro-Mesoporous carbon nitrides for Ultra-High CO₂ and H2S capture. Chemical Engineering Journal, 2023. 456: p. 140973.
[4] Peres, C. B., et al., Advances in Carbon Capture and Use (CCU) Technologies: A Comprehensive Review and CO₂ Mitigation Potential Analysis. Clean technologies, 2022. 4(4): p. 1193-1207.
[5] Ho, H.-J., A. Iizuka, and E. Shibata, Carbon capture and utilization technology without carbon dioxide purification and pressurization: a review on its necessity and available technologies. Industrial & Engineering Chemistry Research, 2019. 58(21): p. 8941-8954.
[6] Gabrielli, P., M. Gazzani, and M. Mazzotti, The role of carbon capture and utilization, carbon capture and storage, and biomass to enable a net-zero-CO₂ emissions chemical industry. Industrial & Engineering Chemistry Research, 2020. 59(15): p. 7033-7045.
[7] Dalena, F., et al., Methanol production and applications: an overview. Methanol, 2018: p. 3-28.
[8] Chein, R.-Y., et al., Analysis of methanol synthesis using CO₂ hydrogenation and syngas produced from biogas-based reforming processes. Chemical Engineering Journal, 2021. 426: p. 130835.
[9] Ting, K. W., et al., Low-temperature hydrogenation of CO₂ to methanol over heterogeneous TiO2-Supported Re catalysts. ACS Catalysis, 2019. 9(4): p. 3685-3693.
[10] Atsbha, T. A., et al., A review on the catalytic conversion of CO₂ using H2 for synthesis of CO, methanol, and hydrocarbons. Journal of CO₂ utilization, 2021. 44: p. 101413.
[11] Doss, B., C. Ramos, and S. Atkins, Optimization of methanol synthesis from carbon dioxide and hydrogen: demonstration of a pilot-scale carbon-neutral synthetic fuels process. Energy & fuels, 2009. 23(9): p. 4647-4650.
[12] Pontzen, F., et al., CO2-based methanol and DME-Efficient technologies for industrial scale production. Catalysis Today, 2011. 171(1): p. 242-250.
[13] Dang, S., et al., A review of research progress on heterogeneous catalysts for methanol synthesis from carbon dioxide hydrogenation. Catalysis Today, 2019. 330: p. 61-75.
[14] Guil-López, R., et al., Methanol synthesis from CO2: a review of the latest developments in heterogeneous catalysis. Materials, 2019. 12(23): p. 3902.
[15] Tisseraud, C., et al., The Cu—ZnO synergy in methanol synthesis from CO₂, Part 2: Origin of the methanol and CO selectivities explained by experimental studies and a sphere contact quantification model in randomly packed binary mixtures on Cu—ZnO coprecipitate catalysts. Journal of Catalysis, 2015. 330: p. 533-544.
[16] Bahruji, H., et al., Pd/ZnO catalysts for direct CO₂ hydrogenation to methanol. Journal of catalysis, 2016. 343: p. 133-146.
[17] Garciá-Trenco, A., et al., PdIn intermetallic nanoparticles for the hydrogenation of CO₂ to methanol. Applied Catalysis B: Environmental, 2018. 220: p. 9-18.
[18] Din, L U., et al., Recent developments on heterogeneous catalytic CO2 reduction to methanol. Journal of CO₂ Utilization, 2019. 34: p. 20-33.
[19] Li, Y., et al., Hydrogenation of CO₂ to methanol over Au—CuO/SBA-15 catalysts. Journal of Porous Materials, 2017. 24: p. 591-599.
[20] Wu, C., et al., Efficient hydrogenation of CO₂ to methanol over supported subnanometer gold catalysts at low temperature. ChemCatChem, 2017. 9(19): p. 3691-3696.
[21] Chen, Y., S. Choi, and L. T. Thompson, Low temperature CO₂ hydrogenation to alcohols and hydrocarbons over Mo2C supported metal catalysts. Journal of catalysis, 2016. 343: p. 147-156.
[22] Joo, O.-S., et al., Carbon dioxide hydrogenation to form methanol via a reverse-water-gas-shift reaction (the CAMERE process). Industrial & engineering chemistry research, 1999. 38(5): p. 1808-1812.
23] Park, S., et al., Camere process for methanol synthesis from C02 hydrogenation. Stud Surf Sci Catal, 2004. 153: p. 67-72.
[24] Stefansson, B. Power and CO₂ emissions to methanol, in Proceedings of the 2015 European Methanol Policy Forum, Brussels, Belgium. 2015.
[25] Chen, C., J. F. K. Kotyk, and S. W. Sheehan, Progress toward commercial application of electrochemical carbon dioxide reduction. Chem, 2018. 4(11): p. 2571-2586.
[26] Adnan, M. A. and M. G. Kibria, Comparative techno-economic and life-cycle assessment of power-to-methanol synthesis pathways. Applied Energy, 2020. 278: p. 115614.
[27] Li, Z. and T. T. Tsotsis, Methanol synthesis in a high-pressure membrane reactor with liquid sweep. Journal of membrane science, 2019. 570: p. 103-111.
[28] Zebarjad, F. S., et al., Experimental investigation of the application of ionic liquids to methanol synthesis in membrane reactors. Industrial & Engineering Chemistry Research, 2019. 58(27): p. 11811-11820.
[29] Dieterich, V., et al., Power-to-liquid via synthesis of methanol, DME or Fischer-Tropsch-fuels: a review. Energy & Environmental Science, 2020. 13(10): p. 3207-3252.
[30] Zebarjad, F. S., et al., Simulation of methanol synthesis in a membrane-contactor reactor. Journal of Membrane Science, 2022. 661: p. 120677.
[31] Gong, J., et al., An experimental and modeling study of the application of membrane contactor reactors to methanol synthesis using pure CO₂ feeds. Chemical Engineering and Processing-Process Intensification, 2023. 183: p. 109241.
[32] Schmidt, O., et al., Future cost and performance of water electrolysis: An expert elicitation study. International journal of hydrogen energy, 2017. 42(52): p. 30470-30492.
[33] Kumar, S. S. and V. Himabindu, Hydrogen production by PEM water electrolysis-A review. Materials Science for Energy Technologies, 2019. 2(3): p. 442-454.
[34] Graaf, G., et al. Kinetics of the three phase methanol synthesis, in Tenth International Symposium on Chemical Reaction Engineering. 1988. Elsevier.
[35] Stefansson, B. Methanol fuel from power and CO₂ emissions. in European Methanol Policy Forum, Brussels. 2015.
[36] Spurgeon, J. M. and B. Kumar, A comparative technoeconomic analysis of pathways for commercial electrochemical CO₂ reduction to liquid products. Energy & Environmental Science, 2018. 11(6): p. 1536-1551.
[37] Küngas, R., electrochemical CO₂ reduction for CO production: comparison of low- and high-temperature electrolysis technologies. Journal of The Electrochemical Society, 2020. 167(4): p. 044508.
[38] Müller, T., et al., Efficient CO₂ to CO electrolysis on solid Ni—N—C catalysts at industrial current densities. Energy & Environmental Science, 2019. 12(2): p. 640-647.
[39] Albo, J., et al., Towards the electrochemical conversion of carbon dioxide into methanol. Green Chemistry, 2015. 17(4): p. 2304-2324.
[40] IRENA, R. E. S., The international renewable energy agency, abu dhabi. Renewable Power Generation Costs in 2019, 2020.
[41] Harris, K., et al., A comparative techno-economic analysis of renewable methanol synthesis from biomass and CO2: Opportunities and barriers to commercialization. Applied Energy, 2021. 303: p. 117637.
[42] Muazzam, Y., et al., Thermo-Economic Analysis of Integrated Hydrogen, Methanol and Dimethyl Ether Production Using Water Electrolyzed Hydrogen. Resources, 2022. 11(10): p. 85.
[43] Kibria, M. G., et al., Electrochemical CO₂ reduction into chemical feedstocks: from mechanistic electrocatalysis models to system design. Advanced Materials, 2019. 31(31): p. 1807166.
[44] Jouny, M., W. Luc, and F. Jiao, General techno-economic analysis of CO2 electrolysis systems. Industrial & Engineering Chemistry Research, 2018. 57(6): p. 2165-2177.
[45] Yang, D., et al., Selective electroreduction of carbon dioxide to methanol on copper selenide nanocatalysts. Nature Communications, 2019. 10(1): p. 677.
[46] Bellotti, D., et al., Feasibility study of methanol production plant from hydrogen and captured carbon dioxide. Journal of CO₂ utilization, 2017. 21: p. 132-138.
[47] Hydrogen Shot-Hydrogen and Fuel Cell Technologies Office. 2021; Available from: https://www.energy.gov/eere/fuelcells/hydrogen-shot.
[48] Sollai, S., et al., Renewable methanol production from green hydrogen and captured CO2: A techno-economic assessment. Journal of CO₂ Utilization, 2023. 68: p. 102345.
[49] Bellotti, D., M. Rivarolo, and L. Magistri, Economic feasibility of methanol synthesis as a method for CO₂ reduction and energy storage. Energy Procedia, 2019. 158: p. 4721-4728.

What is claimed is:

1. A system for converting CO₂ to methanol, the system comprising:
a reverse water gas shift (“RWGS”) reactor configured to receive a first CO₂ stream and a hydrogen gas stream under a sufficient temperature and a sufficient pressure for an RWGS reaction to proceed, the RWGS reactor outputting an exit stream that includes CO;
a heat exchanger/condenser in fluid communication with the RWGS reactor configured to remove water from products of the RWGS reaction to form a dried exit stream that includes CO; and
a membrane contactor reactor configured to receive a combination of hydrogen, CO₂, and the dried exit stream, the membrane contactor reactor further configured to output a first output stream including methanol dissolved in a sweep liquid and a second output stream including gaseous H₂, gaseous CO, gaseous CO₂, and gaseous methanol, the membrane contactor reactor including a tubular ceramic membrane having an interior and an exterior, the membrane contactor reactor further including a packed-bed of methanol synthesis (“MeS”) catalysts surrounding the exterior of the tubular ceramic membrane, the tubular ceramic membrane configured to allow flow of the sweep liquid therethrough such that methanol formed in the packed-bed of MeS catalysts is transported through the tubular ceramic membrane and dissolve in the sweep liquid to form the first output stream, wherein the interior of the tubular ceramic membrane defines a permeate-side and the packed-bed of MeS catalysts defines a reject-side.

2. The system of claim 1, wherein at least a portion of the CO₂ in first CO₂ stream 14 and/or the CO₂ provided to the membrane contactor reactor is derived from flue gas.

3. The system of claim 1, wherein the sweep liquid is a petroleum-derived solvent or an ionic solvent.

4. The system of claim 1, wherein hydrogen content of the exit stream is adjusted via a separate H₂ line.

5. The system of claim 1, wherein the MeS catalysts include a Cu-based MeS catalyst.

6. The system of claim 1, wherein the MeS catalysts include zinc oxide and alumina as a support with copper as an active catalytic component.

7. The system of claim 1, wherein the MeS catalysts include a copper-zinc catalyst.

8. The system of claim 1, further comprising a reject-side back pressure regulator to control the pressure of the reject-side.

9. The system of claim 8, further comprising a reject-side condenser at an outlet of the reject-side to ensure that complete condensation takes place for MeOH, H₂O, and other potential gas phase by-products exiting the membrane contactor reactor.

10. The system of claim 9, wherein the sweep liquid is injected into the permeate-side using a HPLC pump.

11. The system of claim 9, further comprising a permeate-side back pressure regulator in fluid communication with the membrane contactor reactor.

12. The system of claim 11, further comprising a liquid condenser in fluid communication with the permeate-side back pressure regulator and operating at atmospheric pressure and room temperature, to separate any such gas components from the sweep liquid.

13. The system of claim 12, wherein a gas stream from the liquid condenser is recombined with a gas stream exiting reject-side back pressure regulator, a resulting total gas stream then being fed into the reject-side condenser.

14. The system of claim 12, further comprising one or more packed beds below and/or above the packed-bed of MeS catalysts.

15. The system of claim 1, wherein the hydrogen gas stream is produced by an electrolyzer.

16. The system of claim 1 configured to mix the hydrogen gas stream with the first CO₂ stream with a H₂/CO₂ molar ratio of 1:1 to 5:1.

17. The system of claim 1, wherein the sufficient temperature is from about 500° C. to about 700° C.

18. The system of claim 1, wherein the sufficient pressure is from about 0.8 bar to about 3 bar.

19. A method for converting CO₂ to methanol, the method comprising:
providing CO, hydrogen, and CO₂, to a membrane contactor reactor, the membrane contactor reactor configured to output a first output stream including methanol dissolved in a sweep liquid and a second output stream including gaseous H₂, gaseous CO, gaseous CO₂, and gaseous methanol, the membrane contactor reactor including a tubular ceramic membrane having an interior and an exterior, the membrane contactor reactor further including a packed-bed of methanol synthesis (“MeS”) catalysts surrounding the exterior of the tubular ceramic membrane, the tubular ceramic membrane configured to allow flow of the sweep liquid therethrough such that methanol formed in the packed-bed of MeS catalysts is transported through the tubular ceramic membrane and dissolve in the sweep liquid to form the first output stream, wherein the interior of the tubular ceramic membrane defines a permeate-side and the packed-bed of MeS catalysts defines a reject-side; and
separating methanol from the sweep liquid in the first output stream.

20. The method of claim 19, wherein the methanol is separated from the sweep liquid by distillation.
